(19) United States
(12) Patent Application Publication
(10) Pub. No.: US 2024/0307554 A1
(43) Pub. Date: Sep. 19, 2024

(54) TRANSDUCTION BUFFER

(71) Applicants: NTRANS TECHNOLOGIES BV, Utrecht (NL); Koninklijke Nederlandse Akademie van Wetenschappen, Utrecht (NL)

(72) Inventors: Marieke Visscher, Utrecht (NL); Anna Chatsisvili, Utrecht (NL); Axel Beier, Utrecht (NL); Niels Geijsen, Leiden (NL)

(21) Appl. No.: 18/576,991

(22) PCT Filed: Jul. 7, 2022
(86) PCT No.: PCT/EP2022/069006

§ 371 Date: Jan. 5, 2024

(30) Foreign Application Priority Data
Jul. 7, 2021 (GR) 20210100458

(51) Int. Cl. A61K 48/00; A61K 38/46; C12N 9/22; C12N 15/11; C12N 15/90

(52) U.S. Cl. CPC A61K 48/0033; A61K 38/465; C12N 9/22; C12N 15/11; C12N 15/907; C12N 2310/20

(57) ABSTRACT

The invention relates to transduction buffers and methods for transducing molecules into cells using said buffers. The invention also relates to pharmaceutical compositions comprising said transduction buffers and methods of treatment involving the use of said buffers or pharmaceutical compositions to treat diseases, in particular genetic diseases.

### TECHNICAL FIELD

This invention relates to transduction buffers and methods for introducing proteins and nucleic acids into cells using the transduction buffers.

### BACKGROUND

The ability to introduce proteins and nucleic acids into cells has many applications in research and medicine. Unfortunately, the cell membrane presents a major obstacle for the introduction of proteins and nucleic acids into cells. More reliable, non-toxic and efficient methods are needed, which facilitate high transduction efficiencies whilst maintaining cell viability.

Various proteins that have the ability to modify the DNA of mammalian cells have been studied intensively over the past decade. The ability of gene editing proteins to achieve highly specific and readily customizable gene editing in human cells makes such proteins of significant interest for the development of promising therapies for genetic diseases, cancer and infectious diseases. In vivo engineering has been employed for various genetic diseases, such as Duchenne muscular dystrophy (DMD) and Leber's congenital amaurosis (LCA). Ex vivo engineering has found applications in regenerative medicine with the use of modified human induced pluripotent cells (hiPSCs) and in cancer immunotherapy with the use of engineered immune T cells.

The CRISPR/Cas9 gene editing system has been of particular interest to researchers. Its profound ability to delete, integrate or alter genes to create modified cell lines and model organisms across various species has made it a tool of choice for the development of novel therapies. The CRISPR/Cas9 system is comprised of the Cas9 nuclease that induces a site-specific break in the DNA, and a guide RNA (gRNA) that directs Cas9 to a particular site in the genome. The CRISPR/Cas9 system has previously been delivered into cells either in the form of plasmids and viral vectors that subsequently express the Cas9 nuclease and the gRNA, or in the form of a ribonucleoprotein complex (RNP) with the Cas9 nuclease being readily bound to gRNA. The introduction of the CRISPR/Cas9 system into cells using vectors can achieve efficient editing, but the prolonged expression of Cas9 in cells presents a major risk for off-target gene editing. The absence of a simple, fast and efficient method for delivering RNPs such as CRISPR/Cas9 into cells hampers the development of therapeutics for in vivo and ex vivo applications (Lino et al., Drug Deliv., 2018, 25, 1234-1257).

The main delivery methods for gene editing systems such as RNPs into cells in recent years have been microinjection, electroporation and lipofection. Microinjection can achieve high gene-editing efficiency. Electroporation is a method where high-voltage electrical pulses are applied to induce pores in the cell membrane through which RNPs enter the cells and can result in gene editing in difficult-to-transduce cells.

Lipofection is a method where the cargo enters the cells through liposomes and has been used for the cellular delivery of nucleic acid and gene editing systems such as CRISPR/Cas9.

Although each of the above described delivery methods has proven useful in the introduction of gene editing systems into cells, there are major limitations that restrict their applicability in therapeutic settings. Microinjection requires injection of RNPs in individual cells, which is very laborious and thus limits its therapeutic applications only to ex-vivo cell engineering. Electroporation has a negative impact on cell viability, as well as limited applications in vivo due to the requirement to apply a current through patients. Its applications, therefore, are also limited to ex-vivo manipulation of cells. Lipofection is inefficient when targeting therapeutically relevant cell types that are difficult to transduce.

Recently, an additional approach for delivery of a wide range of molecules, including proteins and nucleic acids, into cells was developed. This technology is known as iTOP® (induced transduction by osmocytosis and propanebetaine). This technology involves contacting cells with the molecule to be transduced and a hyperosmotic transduction buffer containing a small molecule transduction compound, such as a propanebetaine or other related molecule, and a group I or “sodium-related” salt. iTOP® exploits a natural cellular uptake process called macropinocytosis, a mechanism employed by the cell to uptake molecules from its surrounding environment. iTOP® has been shown to be a versatile technology for the transduction of a range of different substances into cells. This technology is described in WO 2015/028969 and WO 2017/093326, as well as D'Astolfo et al., Cell. 161, 2015, 674-690.

In the context of protein transduction, the previously reported iTOP® buffers has been shown to achieve efficient modification of a reporter gene upon the delivery of CRE recombinase in different murine cell systems, including mES cells, neural stem cells and gut organoids. Furthermore, reporter gene editing was demonstrated using the iTOP-mediated delivery of the CRISPR/Cas9 system in human KBM7 cells and in human embryonic stem cells, although efficiency was shown to be limited and high concentrations of CRISPR/Cas9 could be required.

There is a need for improved buffers and methods of transduction of proteins and nucleic acids into cells, which combine superior transduction efficiency with high cell viability post-transduction. Efficient transduction of molecules into cells is desirable for a number of therapeutic and scientific purposes, including gene therapy. The provision of improved buffers and methods for transducing proteins and nucleic acids into cells is particularly important to facilitate superior gene editing efficiencies when gene editing proteins are transduced, and thus to provide improved therapies for a wide range of diseases. There is also a particular need to provide methods for transducing proteins and nucleic acids-especially hard-to-transduce proteins such as Cas9-into difficult-to-transduce cell types, such as induced pluripotent stem cells and T cells, in particular human induced pluripotent stem cells (hiPSCs) and primary T cells.

### SUMMARY OF THE INVENTION

The present invention provides a method for transducing a protein, nucleic acid or a combination thereof into a cell, wherein the method comprises contacting said cell with the protein or nucleic acid and a transduction buffer, wherein the transduction buffer comprises:
(i) a transduction compound according to formula I:

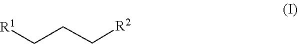

wherein R¹ is selected from SO₃⁻, SO₃H, COOH, COO⁻ or C(O)NR³₂; and
R² is selected from NR³₂, NR³₃⁺,

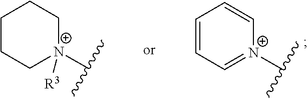

wherein R³ is H or C₁₋₃ alkyl;
(ii) a salt selected from a sodium, rubidium, lithium, potassium or caesium salt, wherein the total concentration of the sodium, rubidium, lithium, potassium or caesium salt is between 500 mM and 1100 mM;
(ii) one or more amino acids selected from serine, asparagine, cysteine, glutamine and threonine, and combinations thereof, wherein the total concentration of amino acids is between 75 mM and 250 mM; and
(iv) one or more disaccharides, wherein the total concentration of disaccharides is between 20 mM and 80 mM.

The method may be performed in vitro or in vivo.

The “method for transducing a protein, nucleic acid or combination thereof” is also referred to herein as the “transduction method” or “method for transduction”. These terms are used interchangeably to refer to the same methods.

The invention also provides a transduction buffer comprising:
(i) a transduction compound according to formula I:

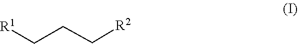

wherein R¹ is selected from SO₃⁻, SO₃H, COOH, COO⁻ or C(O)NR³₂; and
R² is selected from NR³₂, NR³₃⁺,

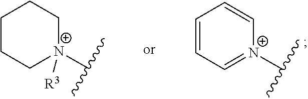

wherein R³ is H or C₁₋₃ alkyl;
(ii) a salt selected from a sodium, rubidium, lithium, potassium or caesium salt, wherein the total concentration of the sodium, rubidium, lithium, potassium or caesium salt is between 500 mM and 1100 mM;
(ii) one or more amino acids selected from serine, asparagine, cysteine, glutamine and threonine, and combinations thereof, wherein the total concentration of amino acids is between 75 mM and 250 mM; and
(iv) one or more disaccharides, wherein the total concentration of disaccharides is between 20 mM and 80 mM.

The invention further provides a pharmaceutical composition comprising the transduction buffer of the invention.

The invention further provides the transduction buffer or the pharmaceutical composition of the invention, for use in therapy.

The invention also provides the transduction buffer or the pharmaceutical composition of the invention, for use in a method of treating a genetic disease, wherein said method comprises contacting a cell with said transduction buffer or pharmaceutical composition and a gene editing protein.

### DETAILED DESCRIPTION OF THE INVENTION

Transduction is the internalisation of molecules into a cell, from the external environment. A small number of proteins and peptides have the inherent property of being able to penetrate the cell membrane. Other proteins can have this transducing property conferred upon them by altering the environmental conditions of the cell or by modifying the protein of interest for transduction.

The invention provides improved methods and buffers for transduction of proteins and nucleic acids into cells. The improved buffer composition allows efficient transduction of proteins and nucleic acids into cells whilst maintaining good cell viability, including facilitating transduction into cells such as induced pluripotent stem cells and T cells, in particular human induced pluripotent stem cells and primary T cells. into which transduction of molecules is notoriously difficult (Chen et al. Cell Stem Cell. 2014, 14, 13-26; Fajrial et al., Theranostics. 2020, 10, 5532-5549; Yu et al Cell Stem Cell. 2015, 16, 142-147). The improved buffers and methods of the invention are particularly useful for transducing large proteins, such as Cas9, and gene editing systems, such as CRISPR/Cas9, into cells. Complexes such as CRISPR/Cas9 are especially challenging to transduce, in part in in view of their low solubility.

It has previously been shown that a transduction buffer comprising a group I or “sodium related” salt, a small molecule transduction compound and, preferably, an osmoprotectant, allows surprisingly efficient uptake of proteins, and other molecules, into cells. The speed and efficiency of the transduction process has been found to depend on the extracellularly applied salt concentration, with higher osmolarities resulting in faster and more efficient uptake. However, the maximum rate of transduction using this method is limited by the fact that buffers with very high osmolarities are detrimental to cell viability and can also affect protein stability and solubility. It has been reported that the replacement of some of the salt with a further non-salt osmolality-inducing component can increase osmolality and thus increase the rate of transduction without damaging cells.

It has now unexpectedly been found that introducing a combination of a disaccharide and an amino acid with a polar neutral side chain into the transduction buffer at particular concentrations leads to improved transduction efficiency of proteins and nucleic acids into cells whilst maintaining good levels of cell viability. Surprisingly, it has also been found that the inclusion of a higher amount of glycerol in addition to the combination of a disaccharide and an amino acid can increase the efficiency of transduction further. Although the inclusion of osmoprotectants such as glycerol in transduction buffers has been described previously for the purpose of preserving cell viability during contact with a hyperosmotic buffer, it has not previously been shown that the addition of a particular concentration of an osmoprotectant such as glycerol can increase the efficiency of transduction of proteins and nucleic acids into cells.

The increased transduction efficiency of the improved buffers means that high levels of transduction can be achieved after a shorter incubation time, thus representing a more convenient method of transduction. Transduction of proteins and nucleic acids into cells can be achieved significantly faster than with previously known methods and buffers. The shorter duration of transduction helps to maintain high cell viability. Additionally, a lower concentration of protein or nucleic acid can be used whilst still achieving efficient transduction. In some instances, the methods and buffers of the invention allow transduction to occur at concentrations as low as 3 μm, which is 5× lower than previously reported.

The improved buffer is particularly useful for transducing large proteins into cells, such as gene editing proteins, including Cas9 proteins. As explained above, the lack of efficient methods of transducing such proteins into cells hampers the development of therapeutic treatments involving such gene editing proteins (Lino et al., Drug Deliv., 2018, 25, 1234-1257). These proteins are difficult to transduce, at least in part due to their low solubility. Surprisingly, the improved buffers and methods of the present invention facilitate improved transduction of proteins into difficult-to-transduce cells such as human induced pluripotent stem cells (hiPSCs) and primary T cells. The invention therefore provides a method for the transduction of proteins which are challenging to transduce, such as Cas9, into difficult-to-transduce cells, thus representing a significant step forward in the field.

The surprising combination of improved transduction efficiency of proteins and nucleic acids and good post-treatment cell viability means that the methods of the invention have the potential to contribute to innovative cell-based therapies for a range of diseases.

### Transduction Compound

Various transduction compounds have been described as suitable for efficient transduction (e.g. see WO 2015/028969 and WO 2017/093326). The compounds of Table 1 are particularly useful for efficient transduction. Thus the transduction buffer of the invention for use in the methods described herein comprises at least one transduction compound as defined below.

The transduction buffer of the present invention comprises a transduction compound according to formula I:

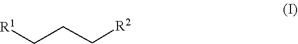

wherein R¹ is selected from SO₃⁻, SO₃H, COOH, COO⁻ or C(O)NR³₂; and
R² is selected from NR³₂, NR³₃⁺,

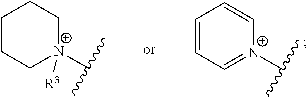

wherein R³ is H or C₁₋₃ alkyl.

Preferably, R¹ is SO₃⁻ or COOH.

Preferably, R² is NR³₂ or

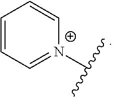 [SMILES: *[n+]1ccccc1]

Preferably, R¹ is SO₃⁻ or COOH and R² is NR³₂ or

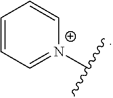 [SMILES: *[n+]1ccccc1]

Table 1 demonstrates that a number of compounds according to formula I are capable of efficient transduction of proteins into cells. Thus, preferred transduction compounds for use in the transduction buffer and methods of the invention include compounds 2, 4, 5, 8, 10, 12, 13, 14, 15, 17, 18, 19, 22, 24, 27 and 30 from Table 1.

Table 1 demonstrates that other compounds besides those of Formula I are effective for transduction of molecules, in particular proteins, into cells. Accordingly, the transduction compound may also be selected from compounds 1, 3, 6, 7, 9, 11, 16, 20, 21, 23, 25, 26, 28, and 29 in Table 1.

Therefore, in one embodiment, the transduction compound is selected from a compound of formula I and compounds 1, 3, 6, 7, 9, 11, 16, 20, 21, 23, 25, 26, 28, and 29 in Table 1. In one embodiment, the transduction compound is selected from compounds 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30 in Table 1, and more preferably from compounds 2, 4, 5, 8, 10, 12, 13, 14, 15, 17, 18, 19, 22, 24, 27 and 30 in Table 1.

As noted above, Table 1 demonstrates that compounds besides those of Formula I are effective for transduction of molecules, in particular proteins, into cells. Accordingly, the transduction compound may be a compound of Formula II:

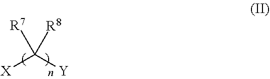

wherein:
X is selected from NR¹R², NR¹R²R³+, OH and COOR⁴;
Y is selected from SO₃H, SO₃⁻, COO⁻, CONH₂, COOR¹², CONR⁵R⁶, tetrazole, OH, NR¹⁰R¹¹, and H;
n is 3, 4, 5 or 6;
R¹, R² and R³, are each independently selected from H, C1-6 alkyl, C5-10 aryl, C6-15 aralkyl, COR⁹; C1-6 alkyl, C5-10 aryl and C6-15 aralkyl may optionally be substituted with R^{Y}, OH or COOH;
or R¹ and R² may come together with the nitrogen to which they are attached to form heterocyclyl;
or when X is NR¹R²R³+, R³ may be absent and R¹ and R² may come together with the nitrogen to which they are attached to form heteroaryl;
R⁴, R⁵, R⁶, R⁹, R¹⁰, R¹¹, R¹² are independently selected from H and C1-6 alkyl;
R⁷ and R⁸ are independently selected from H, C1-6 alkyl and OH;
heterocyclyl is a monocyclic ring which is saturated or partially unsaturated, containing where possible 1 or 2 ring members independently selected from N, NR¹³, NR¹³R¹⁴+ and O, and 2 to 5 carbon atoms; heterocyclyl may optionally be substituted with C1-C6 alkyl, C1-C6 carboxylic acid or C1-C6 alkyl substituted with R^{Y};
heteroaryl is a 5 or 6 membered aromatic ring containing, where possible, 1, 2 or 3 ring members independently selected from N, NR¹³, NR¹³R¹⁴+ and O; heteroaryl may optionally be substituted with C1-C6 alkyl, C1-C6 carboxylic acid or C1-C6 alkyl substituted with R^{Y};
R¹³ and R¹⁴ are independently selected from H, C1-6 alkyl, C1-C6 carboxylic acid and C1-C6 alkyl substituted with R^{Y};
alkyl is a linear or branched saturated hydrocarbon;
R^{Y} is selected from SO₃H, SO₃⁻, COO⁻, CONH₂, COOR¹², CONR⁵R⁶, tetrazole, OH and NR¹⁰R¹¹;
C1-6 carboxylic acid means —COOH or a C1-5 alkyl chain substituted with COOH
and tautomers, solvates, zwitterions and salts thereof.

The transduction compound may also be a compound of formula III:

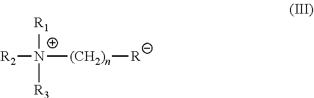

wherein R₁ is methyl, ethyl, propyl, butyl, pentyl or hexyl;
R₂ is methyl, ethyl, propyl, butyl, pentyl or hexyl;
R₃ is methyl, ethyl, propyl, butyl, pentyl or hexyl;
R⁻ is SO₃⁻ or COO⁻ or POO⁻ and
n is 3-6, i.e. 3, 4, 5 or 6.

Accordingly, the present invention provides a method for transducing a protein, nucleic acid or a combination thereof into a cell, wherein the method comprises contacting said cell with the protein or nucleic acid and a transduction buffer, wherein the transduction buffer comprises:
(i) a transduction compound according to formula II:

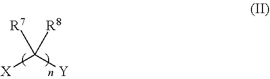

wherein:
X is selected from NR¹R², NR¹R²R³+, OH and COOR⁴;
Y is selected from SO₃H, SO₃⁻, COO⁻, CONH₂, COOR¹², CONR⁵R⁶, tetrazole, OH, NR¹⁰R¹¹, and H;
n is 3, 4, 5 or 6;
R¹, R² and R³, are each independently selected from H, C1-6 alkyl, C5-10 aryl, C6-15 aralkyl, COR⁹; C1-6 alkyl, C5-10 aryl and C6-15 aralkyl may optionally be substituted with R^{Y}, OH or COOH;
or R¹ and R² may come together with the nitrogen to which they are attached to form heterocyclyl;
or when X is NR¹R²R³+, R³ may be absent and R¹ and R² may come together with the nitrogen to which they are attached to form heteroaryl;
R⁴, R⁵, R⁶, R⁹, R¹⁰, R¹¹, R¹² are independently selected from H and C1-6 alkyl;
R⁷ and R⁸ are independently selected from H, C1-6 alkyl and OH;
heterocyclyl is a monocyclic ring which is saturated or partially unsaturated, containing where possible 1 or 2 ring members independently selected from N, NR¹³, NR¹³R¹⁴+ and O, and 2 to 5 carbon atoms; heterocyclyl may optionally be substituted with C1-C6 alkyl, C1-C6 carboxylic acid or C1-C6 alkyl substituted with R^{Y};
heteroaryl is a 5 or 6 membered aromatic ring containing, where possible, 1, 2 or 3 ring members independently selected from N, NR¹³, NR¹³R¹⁴+ and O; heteroaryl may optionally be substituted with C1-C6 alkyl, C1-C6 carboxylic acid or C1-C6 alkyl substituted with R^{Y};
R¹³ and R¹⁴ are independently selected from H, C1-6 alkyl, C1-C6 carboxylic acid and C1-C6 alkyl substituted with R^{Y};
alkyl is a linear or branched saturated hydrocarbon;
R^{Y} is selected from SO₃H, SO₃⁻, COO⁻, CONH₂, COOR¹², CONR⁵R⁶, tetrazole, OH and NR¹⁰R¹¹;
C1-6 carboxylic acid means —COOH or a C1-5 alkyl chain substituted with COOH
and tautomers, solvates, zwitterions and salts thereof;
or a transduction compound according to formula III:

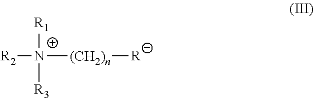

wherein R₁ is methyl, ethyl, propyl, butyl, pentyl or hexyl;
R₂ is methyl, ethyl, propyl, butyl, pentyl or hexyl;
R₃ is methyl, ethyl, propyl, butyl, pentyl or hexyl;
R⁻ is SO₃⁻ or COO⁻ or POO⁻ and
n is 3-6, i.e. 3, 4, 5 or 6;
(ii) a salt selected from a sodium, rubidium, lithium, potassium or caesium salt, wherein the total concentration of the sodium, rubidium, lithium, potassium or caesium salt is between 500 mM and 1100 mM;
(ii) one or more amino acids selected from serine, asparagine, cysteine, glutamine and threonine, and combinations thereof, wherein the total concentration of amino acids is between 75 mM and 250 mM; and
(iv) one or more disaccharides, wherein the total concentration of disaccharides is between 20 mM and 80 mM.

All features which are disclosed herein in preferred embodiments and/or dependent claims in relation to a method of transduction or transduction buffer comprising a transduction compound of formula I apply equally to methods and buffers comprising a compound of formula II or formula III.

It is preferred that the transduction compound is a compound of Formula I or another compound from Table 1, more preferably a compound of Formula I.

Particularly preferred transduction compounds for use in the transduction buffer and methods of the invention are compound 12 (GABA) and compound 22 (NDSB-201). Most preferably, the transduction compound is GABA.

Where transduction compounds according to formula I bear a net positive or negative charge, a counterion is also present. The present invention thus includes salts of the transduction compounds of formula I. When the transduction compound is positively charged, examples of the counterion include chloride, bromide, iodide and hydroxide.

The concentration of the transduction compound in the transduction buffer is not particularly limited. It is to be understood that the optimum concentration of transduction compound will depend on the compound and its efficiency in promoting transduction, as well as the solubility and other properties of the protein or nucleic acid to be transduced. The most suitable concentration of transduction compound to use may be readily determined by the person skilled in the art, for example using the experiments described in the examples.

The concentration of the transduction compound according to formula I in the transduction buffer may be from 100 mM to 600 mM, from 100 mM to 500 mM, from 100 mM to 400 mM, from 200 mM to 300 mM, or from 230 mM to 270 mM. Preferably, the concentration of the transduction compound is from 100 mM to 400 mM, more preferably from 200 mM to 300 mM. Most preferably, the concentration of the transduction compound is about 250 mM.

In a preferred embodiment, the transduction compound is GABA or NDSB-201 and the concentration of the transduction compound is from 100 mM to 600 mM, from 100 mM to 500 mM, from 100 mM to 400 mM, from 200 mM to 300 mM, or from 230 mM to 270 mM. Preferably, the transduction compound is GABA or NDSB-201 and the concentration of the transduction compound is from 200 mM to 300 mM.

In a further preferred embodiment, the transduction compound is GABA and the concentration of GABA is from 100 mM to 600 mM, from 100 mM to 500 mM, from 100 mM to 400 mM, from 200 mM to 300 mM, or from 230 mM to 270 mM. Most preferably, the transduction compound is GABA and the concentration of GABA is from 200 mM to 300 mM.

In some embodiments, one transduction compound is included in the transduction buffer. In some embodiments, more than one transduction compound is included in the transduction buffer, for example, two, three, four or five transduction compounds.

### Salt in the Transduction Buffer

A “sodium-related” salt, i.e. a salt containing a metal in group 1 of the periodic table, in a transduction buffer in combination with a small molecule transduction compound is useful in the transduction buffer. In general the salt cannot freely diffuse across a cell membrane and thus generates hypertonicity by increasing the tonicity across the cell membrane when brought into contact with a cell.

The salt in the transduction buffer of the invention is a sodium, lithium, potassium, caesium, or a rubidium salt. Preferably, the salt is a sodium salt. Preferably, the salt is a sodium salt and no lithium, potassium, caesium, or rubidium salts are present in the transduction buffer.

In some embodiments, the salt is a chloride, gluconate, carbonate, sulphonate, sulphate, sulphide, bromide, iodide or fluoride salt, preferably a chloride or gluconate. Non-limiting examples of salts suitable for use in the transduction buffer include sodium chloride, sodium gluconate, lithium chloride, lithium gluconate, potassium chloride, potassium gluconate, caesium chloride, caesium gluconate, rubidium chloride and rubidium gluconate. Most preferably, the salt is sodium chloride.

The total concentration of the sodium, lithium, potassium, caesium, or rubidium salt in the transduction buffer is from 500 mM to 1100 mM. In some embodiments, the total concentration of the sodium, lithium, potassium, caesium, or rubidium salt in the transduction buffer is from 550 mM to 1050 mM, from 600 mM to 1000 mM, from 650 mM to 950 mM, from 700 mM to 900 mM, from 750 mM to 850 mM or from 780 mM to 820 mM. Preferably, the total concentration of the sodium, lithium, potassium, caesium, or rubidium salt in the transduction buffer is from 700 mM to 900 mM, more preferably from 750 mM to 850 mM. Most preferably, the total concentration of the sodium, lithium, potassium, caesium, or rubidium salt in the transduction buffer is about 800 mM.

In some preferred embodiments, the salt is sodium chloride and the concentration of sodium chloride is from 550 mM to 1050 mM, from 600 mM to 1000 mM, from 650 mM to 950 mM, from 700 mM to 900 mM, from 750 mM to 850 mM or from 780 mM to 820 mM. Preferably, the concentration of sodium chloride is from 700 mM to 900 mM, more preferably from 750 mM to 850 mM. Most preferably, the total concentration of sodium chloride in the transduction buffer is about 800 mM. It is particularly preferred that sodium chloride is the only sodium, lithium, potassium, caesium or rubidium salt present in the buffer in a concentration of more than 10 mM.

In some embodiments, one sodium, lithium, potassium, caesium, or a rubidium salt is included in the transduction buffer. In some embodiments, more than one sodium, lithium, potassium, caesium, or a rubidium salt is included in the transduction buffer, for example, two, three, four or five salts. Preferably, no lithium, potassium, caesium, or rubidium salts are present in the transduction buffer. Preferably, only one sodium-related salt is included in the buffer at a concentration of greater than 10 mM, most preferably wherein that salt is sodium chloride.

### The Disaccharide and Amino Acid in the Transduction Buffer

It has surprisingly been found that a combination of a disaccharide and an amino acid with a polar neutral side chain at particular concentrations in a transduction buffer containing a transduction compound and a group I salt leads to increased transduction efficiency of proteins and nucleic acids into cells. It also enables the particularly efficient transduction of proteins and nucleic acids into difficult-to-transduce cells, such as induced pluripotent stem cells and T cells, in particular hiPSCs and primary T cells.

Thus, the transduction buffer of the invention for use in the methods disclosed herein comprises one or more disaccharides and one or more amino acids.

In some embodiments, the disaccharide is selected from trehalose, sucrose, lactose, maltose, cellobiose and lactulose, and combinations thereof. Preferably, the disaccharide is trehalose.

The total concentration of disaccharides in the transduction buffer is between 20 mM and 80 mM. Preferably, the total concentration of disaccharides in the transduction buffer is between 40 mM and 60 mM, most preferably about 50 mM.

In a preferred embodiment, the disaccharide is trehalose, wherein the total concentration of trehalose is between 20 mM and 80 mM, more preferably between 40 mM and 60 mM and most preferably about 50 mM.

The amino acid has a polar neutral side chain. Thus, the amino acid is selected from serine, asparagine, cysteine, glutamine and threonine, and combinations thereof. In some embodiments, the amino acid is selected from serine, asparagine, cysteine, and threonine, and combinations thereof. In some embodiments, the amino acid is selected from serine, threonine, and combinations thereof. Preferably, the amino acid is serine.

In a particularly preferred embodiment, the disaccharide is trehalose and the amino acid is serine.

The total concentration of amino acids in the buffer is between 75 mM and 250 mM. Preferably, the total concentration of amino acids in the buffer is between 100 mM and 200 mM, more preferably between 125 mM and 175 mM and most preferably about 150 mM.

Preferably, the total concentration of disaccharides in the transduction buffer is between 40 mM and 60 mM and the total concentration of amino acids in the buffer is between 100 mM and 200 mM, more preferably between 125 mM and 175 mM.

In a preferred embodiment, the transduction buffer comprises serine and the total concentration of serine in the buffer is between 75 mM and 250 mM. More preferably, the total concentration of serine is between 100 mM and 200 mM, most preferably between 125 mM and 175 mM, for example about 150 mM.

In a further preferred embodiment, the transduction buffer comprises trehalose and serine, wherein the total concentration of trehalose is between 40 mM and 60 mM and the total concentration of serine is between 100 mM and 200 mM. For example, the total concentration of trehalose may be about 50 mM and the total concentration of serine may be about 150 mM.

### Osmolality of the Transduction Buffer

Osmolality is the concentration of a solution in terms of osmoles of solutes per kilogram of solvent. It differs from osmolarity which is the concentration of osmoles of solutes per volume of solvent. Osmolarity is temperature dependent because water changes its volume with temperature. Therefore, osmolality is the preferred measure because it is not temperature dependent. If the concentration of solutes is very low, osmolarity and osmolality are considered equivalent.

Tonicity, by contrast, is defined by the concentration of all solutes that do not cross a cell membrane, i.e. the concentration of solutes that result in osmotic pressure across a cell membrane. In the context of the transduction buffer, hyperosmolality is achieved using hypertonic salts in combination with other components such as disaccharides and amino acids, as described above. For the transduction method to work, it is important that there is osmotic pressure across the cell membrane. Thus, whilst the transduction buffer can be defined by osmolality (in isolation of the cell), the method of transduction requires the transduction buffer to be hypertonic with respect to the cell cytosol. A cell placed in a hypertonic solution. such as a transduction buffer described herein, will lose water by osmosis. This causes the cell to shrink and tends to increase the space in between cells in a population. To compensate for the loss in cell volume. the cells activate macropinocytosis, i.e. the influx of macromolecules from the extracellular environment. It is to be understood that the optimum osmolality of the transduction buffer is cell-type specific and is defined, in part, by the osmolality of the culture media used to maintain the cell prior to transduction and/or the osmolality of the cell cytosol.

Thus in some embodiments, the method for transducing a molecule of interest into a cell involves the step of increasing the osmotic pressure outside of the cell. In some embodiments, there is osmotic pressure across the cell membrane. In some embodiments, the transduction buffer is hypertonic with respect to the culture media in which the cell was maintained prior to transduction and/or with respect to the cell cytosol. In other words, the osmolality of the transduction buffer is greater than the osmolality of the culture media in which the cell was maintained prior to transduction and/or greater than the cell cytosol.

Normal osmolality of human serum is about 275-295 mOsm/kg. While temporary elevation of serum osmolality has been used to reduce brain edema in stroke patients, prolonged elevated global osmolality in a human can lead to complications and in serious cases can be fatal. For this reason, pharmaceutical compositions are typically isotonic (have approximately the same osmolality as serum). Individual cells. however, can survive at much higher osmolalities (e.g. up to about 1000 mOsm/kg). Thus, live organisms are able to tolerate moderate elevation of osmolality for several days and temporary high osmolalities locally.

Hyperosmolality refers to an abnormal increase in the osmolality of a solution, especially a body fluid or culture medium. The osmolality at which human cells are maintained is typically about 275-295 mOsm/Kg but, for example, preimplantation embryos are grown at an osmolality of about 250-260 mOsm/Kg. Therefore, in the context of a typical human cell, hyperosmolality refers to an osmolality of more than about 250 mOsm/kg. Thus a transduction buffer with an osmolality of more than about 295 mOsm/kg is likely to be hypertonic with respect to a typical human cell, whereas a transduction buffer with an osmolality of more than about 260 mOsm/kg is likely to be hypertonic with respect to early embryos. Hypo-osmolality refers to an abnormal decrease in the osmolality of a solution, especially a body fluid. Therefore, in the context of typical human cells hypo-osmolality refers to an osmolality of less than about 295 mOsm/kg. Thus a transduction buffer with a tonic salt-mediated osmolality of less than about 295 mOsm/kg is likely to be hypotonic with respect to a typical human cell. In the context of a typical embryo, hypo-osmolality refers to an osmolality of less than about 260 mOsm/kg. Thus a transduction buffer with a tonic salt-mediated osmolality of less than about 260 mOsm/kg is likely to be hypotonic with respect to a typical early embryo.

The osmolality of the transduction buffer can be determined by methods known in the art using an osmometer or can be calculated, e.g. if the osmolar pressure of the media which makes up the remaining volume of the buffer is known. Thus, the salt or further osmolality-inducing component can be added to adjust the osmolality of the buffer to the desired level.

In one embodiment, the transduction buffer has an osmolality of at least 1500 mOsm/kg, preferably at least 1800 mOsm/kg, more preferably at least 2000 mOsm/kg.

Typically, the transduction buffer of the invention has an osmolality of between 2000 mOsm/kg and 4500 mOsm/kg, more typically between 2000 mOsm/kg and 4000 mOsm/kg.

In one embodiment, the transduction buffer has an osmolality of between 2000 mOsm/kg and 2500 mOsm/kg, preferably between 2000 mOsm/kg and 2250 mOsm/kg. Such buffers are particularly useful for transducing gene editing proteins such as Cas proteins, into cells. Therefore, in one embodiment, there is provided a method for transducing a gene editing protein, preferably a Cas protein and more preferably a Cas9 protein, into a cell, by contacting the cell with a transduction buffer having an osmolality of between 2000 mOsm/kg and 2500 mOsm/kg, more preferably between 2000 mOsm/kg and 2250 mOsm/kg, and a gene editing protein.

In one embodiment, the transduction buffer has an osmolality of between 3000 mOsm/kg and 4000 mOsm/kg, preferably between 3500 mOsm/kg and 4000 mOsm/kg.

The data in the examples shows that in some instances, a buffer having a higher osmolality (i.e. NT2100.2) demonstrates improved transduction efficiency relative to a buffer having a lower osmolality (i.e. NT2100). Therefore, in a preferred embodiment, the transduction buffer has an osmolality of between 3000 mOsm/kg and 4000 mOsm/kg, more preferably between 3500 mOsm/kg and 4000 mOsm/kg. The use of this transduction buffer leads to further improved transduction efficiency of proteins and nucleic acids into cells, whilst maintaining excellent cell viability. Such high osmolality buffers are especially useful for transducing poorly soluble proteins such as Cas9 proteins, into cells.

The use of a transduction buffer having an osmolality of between 3000 mOsm/kg and 4000 mOsm/kg, more preferably between 3500 mOsm/kg and 4000 mOsm/kg, is shown to be particularly effective for transducing gene editing proteins such as Cas proteins into cells. Therefore, in a preferred embodiment, there is provided a method for transducing a gene editing protein, preferably a Cas protein and more preferably a Cas9 protein, into a cell, by contacting the cell with a transduction buffer having an osmolality of between 3000 mOsm/kg and 4000 mOsm/kg, more preferably between 3500 mOsm/kg and 4000 mOsm/kg, and a gene editing protein.

### The Osmoprotectant for Transduction

The osmolality of the transduction buffer is such that during transduction methods the transduction buffer is hypertonic with respect to the cell. This can cause osmotic stress to cells and in certain circumstances this can reduce cell proliferation or viability. Addition of osmoprotectants can protect against these effects, as disclosed in WO2015/028969.

Osmoprotectants are small molecules that act as osmolytes and help protect cells and organisms from osmotic stress. Chemically, osmoprotectants can be divided into three types: betaines and allied compounds, polyols and sugars (e.g. glycerol), and amino acids. Betaines are methyl derivatives of glycine in which the nitrogen atom is fully methylated, i.e. they are quaternary ammonium compounds. Other methyl derivatives of glycine useful in the context of this invention include, but are not limited to, sarcosine and dimethylglycine. It will be clear to the skilled person that some of the transduction compounds described herein can thus function as osmoprotectants. A non-limiting example of a transduction compound that also functions as an osmoprotectant is GABA. However, not all osmoprotectants enhance transduction. Similarly, not all transduction compounds function as osmoprotectants. Therefore, in some embodiments an osmoprotectant is added to the transduction buffer in addition to the transduction compound (which may or may not function as an osmoprotectant in this context).

The transduction buffer of the invention preferably comprises an osmoprotectant.

In some embodiments, the osmoprotectant is selected from glycine, glycerol, taurine, glycinebetaine, myo-inositol, arginine and mannitol. In a preferred embodiment, the osmoprotectant is glycine and/or glycerol.

In some embodiments, the transduction buffer comprises more than one type of osmoprotectant. Most preferably, the osmoprotectant is glycine and glycerol. Glycine and glycerol is particularly suitable for use with murine embryonic fibroblast cells, embryonic stem cells, human iPS cells and primary T cells. However, any combination of osmoprotectants may be suitable for use in the transduction buffer of the invention. For example, any combination of osmoprotectants described herein, for example any combination of 2, 3, 4, 5, 6, 7 or all of glycine, glycerol, taurine, glycinebetaine, myo-inositol, arginine and mannitol.

The type (or combination of types) of osmoprotectant selected for use with the invention may depend upon the type of cell to be transduced. The suitability of an osmoprotectant can be easily determined by the skilled person by assays known in the art.

The concentration of osmoprotectant selected for use with the invention is not particularly limited and may depend upon the type of cell to be transduced. In some embodiments, the osmoprotectant is at a concentration of between about 10 mM and 2000 mM or between 30 mM and 1700 mM.

In some embodiments, the concentration of osmoprotectant is between 30 mM and 60 mM, preferably between 40 mM and 60 mM. More preferably, the osmoprotectant is glycine and glycerol at a total concentration of about 45 mM, for example about 15 mM glycine and 30 mM glycerol.

It has surprisingly been found that the addition of a higher amount of glycerol leads to improved transduction efficiency. As explained above, the inclusion of an osmoprotectant in a transduction buffer has been described previously to protect cell viability, but it has not been suggested that the efficiency of transduction could be increased by increasing the concentration of an osmoprotectant.

In some preferred embodiments, the transduction buffer comprises glycerol at a concentration of between 1000 mM and 2000 mM. Preferably, the glycerol is at a concentration of between 1300 mM and 1700 mM, most preferably between 1400 mM and 1600 mM. It has unexpectedly been found that the presence of glycerol at these elevated concentrations can improve the efficiency of proteins and nucleic acids into cells relative to the inclusion of lower concentrations of glycerol, e.g. below 1000 mM. Therefore, the use of a glycerol concentration of between 1400 mM and 1600 mM is especially preferable for increasing the efficiency of transduction.

### Other Components of the Transduction Buffer

It is to be understood that the any of the additional components of the transduction buffer described herein may be part of the transduction buffer. Alternatively, they may be added simultaneously or sequentially to the cells in any combination as a step in the method of transduction.

The transduction buffer may additionally comprise components that make it particularly suitable for use with live cells or live cell culture or application in vivo. For example, in some embodiments the transduction buffer comprises one or more of a biological pH buffer, a viscosity enhancer, and/or one or more growth factor(s), vitamins and nutrients.

A transduction buffer of the invention will normally be formulated in deionized, distilled water, although suitable alternatives may be used including, but not limited to cell culture media or therapeutic solutions. It will typically be sterilized prior to use to prevent contamination, e.g. by ultraviolet light, heating, irradiation or filtration. It may be frozen (e.g. at between −20° C. or −80° C., for examples at −20° C. or at −80° C.) for storage or transport. The transduction buffer may contain one or more antibiotics, such as doxycycline or tetracycline, to prevent contamination. However, some antibiotics, particularly non cell-permeable antibiotics (such as penicillin and/or streptomycin), can be toxic to the cells when transduced into the cells. Therefore, in some embodiments, the transduction buffer does not comprise an antibiotic, for example the transduction buffer does not comprise a non cell-permeable antibiotic. In some embodiments. the transduction buffer does not comprise penicillin.

The transduction buffer may be buffered by a biological pH buffer at a pH of between about 6 and about 8. for example a pH of between about 7.2 and about 7.6 or a pH of about 7.4. A pH outside of this range (i.e. higher than 8 or lower than 6) might be appropriate for administration to particular tissues, as would easily be determined by the person skilled in the art. For example, stomach pH can drop to as low as 1 or 2. Therefore, a transduction buffer for administration to the stomach may have a pH of less than 7, less than 6, less than 5, less than 4, less than 3, less than 2, for example a pH of 7, 6, 5, 4, 3, 2 or 1. A biological pH buffer is a pH buffer that is suitable for use with live cells, i.e. which has minimal negative impact on cell viability. The biological pH buffer may be a phosphate based buffer or any other suitable buffer. A number of biological pH buffers are known in the art (see for example the biological buffers provided in Plant Microtechnique and Microscopy. Oxford University Press. Steven E. Ruzin. ISBN: 0-19-508956-1; and www.sigmaaldrich.com/life-science/core-bioreagents/biological-buffers/biological-buffer-products.html). Examples of biological pH buffers include, but are not limited to PBS. TES. TRIS, PIPES. MOPS, MES. Good's buffers. Trizma or HEPES. Thus in some embodiments the transduction buffer additionally comprises PBS. TES. TRIS. PIPES. MOPS. MES. Good's buffers. Trizma or HEPES. Some of the transduction compounds are also excellent buffering compounds, so can act as buffers instead of, or in addition to, the biological buffer.

The transduction buffer may be supplemented with purified, natural, recombinant, semi-synthetic and/or synthetic growth factors. Any suitable growth factor or combination of growth factors may be used. Non-limiting examples of suitable growth factors include EGF. FGF. HGF. PDGF. BDNF. VEGF or IGF. Any combination of suitable growth factors may be used. Non-limiting examples of growth factor combinations include any one or more (e.g. 1, 2, 3, 4, 5 or 6) of the growth factors in the list consisting of: EGF, FGF (e.g. FGF2, FGF7 or FGF10), HGF, PDGF, BDNF, VEGF or IGF. The growth factors added may, in some circumstances depend on the cell to be transduced, and it is known in the art how to select appropriate growth factors for a particular cell.

The growth factor or growth factors is preferably added at a concentration of between about 1 and about 500 ng/ml or of at least 5 and not higher than 500 ng/ml. A preferred concentration is at least 1, 2, 5, 10, 20, 25, 30, 40, 45, 50, 60, 70, 80, 90, 100, 200, 300, 400 ng/ml and not higher than 600, 500, 450, 400, 350, 300, 250, 200, 150, or 100 ng/ml. A more preferred concentration is at least 10 ng/ml and not higher than 500 ng/ml. An even more preferred concentration is about 50 ng/ml or about 100 ng/ml. The skilled person will be aware that the optimal concentration of a growth factor is both dependent upon the growth factor and the cell to be transduced. The optimal concentration can be determined by methods known in the art and by the methods described in the examples herein.

In some embodiments, the transduction buffer is supplemented with a cytokine. Similarly, to growth factors, different cytokines are suitable for the culture of different cell types and suitable cytokines are known in the art. Other cell type specific factors known in the art can also be added to the transduction buffer, such as, but not limited to LIF (for maintaining the stem cell state of embryonic stem cells) and GM-CSF for dendritic cells.

In some embodiments the transduction buffer additionally comprises a viscosity enhancer. This is particularly preferred when the transduction buffer is for use in vivo because it prevents unwanted dispersion of the transduction buffer. This, therefore, helps to keep the buffer in contact with the cells being transduced. In some embodiments, the viscosity enhancer is polyvinylpyrrolidone (PVP), polyvinyl alcohol, methylcellulose, hydroxypropylmethylcellulose, hydroxyethylcellulose, sodium carboxymethyl cellulose (NaCMC), propylene glycol alginate (PGA) or sodium alginate (SA). A preferred viscosity enhancer is non-toxic and suitable for use with live cells and/or in vivo.

In some embodiments, the transduction buffer additionally comprises an antioxidant, such as ethylenediaminetetraacetic acid (EDTA), sodium bisulfite, sodium metabisulfite, ascorbic acid or thiourea.

In some embodiments, the transduction buffer additionally comprises a basal culture medium. Suitable culture media are available commercially, and include, but are not limited to, Optimem, Dulbecco's Modified Eagle Media (DMEM), Minimal Essential Medium (MEM), Knockout-DMEM (KO-DMEM), Glasgow Minimal Essential Medium (G-MEM), Basal Medium Eagle (BME), DMEM/Ham's F12, Advanced DMEM/Ham's F12, Iscove's Modified Dulbecco's Media and Minimal Essential Media (MEM), Ham's F-10, Ham's F-12, Medium 199, and RPMI 1640 Media.

In some embodiments, the transduction buffer additionally comprises serum. However, in a preferred embodiment, the transduction buffer does not comprise an undefined component such as fetal bovine serum or fetal calf serum. Various different serum replacement formulations are commercially available and are known to the skilled person. Where a serum replacement is used, it may for example be used at between about 0.1% and about 50% by volume of the medium, according to conventional techniques.

Transduction is typically performed in culture medium that is appropriate for the regular maintenance of the particular cell type. As with any of the factors described herein, this culture medium may be part of the transduction buffer or it may be added to the cells separately in the transduction method. In a preferred embodiment, there is no serum or a reduced concentration of serum in the culture medium used during transduction.

The concentration ranges provided for all components of the buffer are final concentrations when the buffer is in use for transduction (e.g. concentrations when the buffer is formulated in deionized, distilled water, cell culture medium or a therapeutic composition).

Proteins for transduction are typically provided in a 5× or 10× concentrate, preferably a 5× concentrate, which when added to the cell culture media gives the concentrations described herein.

### Methods for Transduction

The invention provides a method for transducing a protein or nucleic acid into a cell, wherein the method comprises the steps of contacting said cell with the protein or nucleic acid and contacting said cell with a transduction buffer of the invention.

In some embodiments, the invention provides a method for transducing a protein or nucleic acid into a cell, wherein the method comprises the steps of contacting said cell with the protein or nucleic acid and contacting said cell with a transduction buffer of the invention, thereby transducing the protein or nucleic acid into the cell. In some embodiments, the invention provides a method for transducing a gene editing protein into a cell, wherein the method comprises the steps of contacting said cell with the gene editing protein and contacting said cell with a transduction buffer of the invention, thereby transducing the gene editing protein into the cell.

The protein or nucleic acid and the transduction buffer are contacted with the cell in combination, either simultaneously, sequentially, or separately in any order. In a preferred embodiment, they are administered simultaneously (e.g. from a container containing the combination). Thus, in some preferred embodiments, the transduction buffer comprises the protein or nucleic acid. In some embodiments, the method involves the step of mixing the transduction buffer and the protein or nucleic acid.

In some embodiments, the method comprises the step of obtaining and/or maintaining the cells in culture medium prior to transduction. In some embodiments, the cell is plated in a culture medium, suitable for the particular cell, prior to transduction. In some embodiments, the method further comprises contacting the cell with a culture medium during transduction. In some embodiments, the method includes the step of mixing the transduction buffer with a culture medium.

In some embodiments, the method comprises the steps of obtaining the cells and maintaining the cells in culture medium prior to transduction and contacting the cell with culture medium during transduction. In some embodiments, the method includes the step of mixing the transduction buffer with a culture medium prior to contacting the cell with the transduction buffer. In some embodiments, after transduction, the transduction buffer is aspirated and/or the cells are washed, e.g. once or twice. Typically, a regular culture medium, suitable for the particular cell type, will be added to the cells at this stage. In some embodiments, the method comprises the step of obtaining the cells and/or maintaining the cells in culture medium after transduction.

The method for transduction may be performed in vivo or in vitro.

In some embodiments, the transduction method does not involve a transmembrane carrier, for example selected from a viral plasmid, a nanoparticle, a liposome or other lipid vesicle (including micelles). In some embodiments, the transduction method is non-viral, meaning that it does not rely on a viral transfection system and/or does not involve a viral plasmid, for example as a transmembrane carrier. In some embodiments the transduction method does not involve cationic lipids, for example as transmembrane carriers. In some embodiments, the transduction method does not involve liposomes, for example as transmembrane carriers. In some embodiments, the transduction method does not involve nanoparticles, for example as transmembrane carriers. In some embodiments, the transduction method does not involve outer membrane vesicles (OMVs), for example as transmembrane carriers. In some embodiments the methods does not involve cell penetrating peptides. In a preferred embodiment, the transduction method does not involve exposure of the cell to a hypotonic environment, e.g. a hypotonic buffer.

In some embodiments, the method involves activating or enhancing macropinocytosis and/or enhancing endosomal lysis, thus enhancing uptake of molecules, particularly the molecule of interest, into the cell. In the context of this application, it is to be understood that “endosomes”, which are internal invaginations of the cell membrane involved in macropinocytosis, and comprising a complex mixture of lipids, differ from “liposomes” or “micelles”, which are synthetic lipid vesicles typically formed from a fewer types of lipid molecule, and from “OMVs”, which are bacterial vesicles which may be modified to make them suitable as transmembrane carriers.

In one embodiment (NT2100) the transduction buffer comprises GABA (e.g. 250 mM); sodium chloride (e.g. 800 mM); trehalose (e.g 50 mM); serine (e.g. 150 mM); glycine (e.g. 15 mM) and glycerol at a concentration of 10-50 mM (e.g. 30 mM). In addition, the transduction buffer may comprise culture medium components, such as non-essential amino acids, N2, B27, phosphate buffer, and a basal medium (e.g. a reduced serum medium, e.g. Optimem).

In another embodiment (NT2100.2) the transduction buffer comprises GABA (e.g. 250 mM); sodium chloride (e.g. 800 mM); trehalose at a concentration of 50 mM; serine at a concentration of 150 mM; glycine (e.g. 15 mM) and glycerol at a concentration of 1300-1700 mM (e.g. 1530 mM). In addition, the transduction buffer may comprise culture medium components, such as non-essential amino acids, N2, B27, phosphate buffer, and a basal medium (e.g. a reduced serum medium, e.g. Optimem).

In some embodiments, the day before (e.g. about 12 to 24 hours before) transduction, cells are plated in the appropriate culture media without antibiotics. The following day (the day of transduction), the transduction buffer is prepared with the protein or nucleic acid. The transduction buffer and the protein or nucleic acid are mixed with cell culture medium to obtain a transduction buffer with the desired composition. This mixture of media/transduction buffer/protein or nucleic acid is added to the cell. The cell is incubated with the protein or nucleic acid in the transduction buffer for at least the necessary time period for transduction to occur, after which time, the transduction media is removed and exchanged for regular culture media.

For the avoidance of any doubt, it is to be understood that these methods and protocols are compatible and combinable with the transduction compounds, salts, disaccharides, amino acids, osmoprotectants, and other additional components, and concentrations thereof, of the transduction buffer described in detail above. These protocols and methods can be used to transduce various proteins and nucleic acids into cells. including gene editing proteins that are challenging to transduce into cells.

### Cell Viability

As discussed elsewhere, the ability to transduce proteins and nucleic acids into cells has many applications in research and medicine. However, known methods for cell transduction, such as electroporation can have a negative impact on cell viability. The inventors have surprisingly discovered that the transduction buffer and methods of the invention can facilitate high transduction efficiencies of proteins and nucleic acids whilst maintaining good levels of cell viability. This is aided by the increased transduction efficiency of the improved transduction buffer of the invention, which means a shorter duration of transduction can be used. even relative to previously described buffers containing a transduction compound and a sodium-related salt. This helps to maintain high cell viability. In a preferred embodiment, the transduction buffer and methods of the invention have minimal impact on the viability of the cells. Cell viability is important for many applications of the transduced cells, including but not limited to transplantation of transduced cells; the use of transduced cells to generate genetically modified embryos for research models; and the use of transduced cells in research etc.

Assays to measure proliferation, viability and cytotoxicity are known in the art and available commercially (e.g. from Sigma Aldrich). Such assays can be used to monitor the response and health of cells in culture after treatment with various stimuli. The proper choice of an assay method depends on the number and type of cells used as well as the expected outcome. One measure of cell viability is cellular proliferation (e.g the BrdU incorporation assay). Continuing cellular proliferation demonstrates that the normal cell cycle is still functioning. Assays for cell proliferation may monitor the number of cells over time, the number of cellular divisions, metabolic activity or DNA synthesis. Cell counting using viability dyes such as trypan blue or calcein-AM can provide both the rate of proliferation as well as the percentage of viable cells. Viability can also be determined using FACS (fluorescence-activated cell sorting) based assays, for example using FACS to count unstained/stained cells treated with dead cell stains (e.g. SYTOX Red). 5(6)-Carboxyfluorescein diacetate N-succinimidyl ester (CFSE) is a popular choice for measuring the number of cellular divisions a population has undergone. Upon entering the cell. CFSE is cleaved by intracellular esterases to form the fluorescent compound and the succinimidyl ester group covalently reacts with primary amines on intracellular proteins. Upon division, the fluorescence intensity of each daughter cell is halved which allows for the simple detection of the number of cell divisions by flow cytometry. Assays that measure metabolic activity are suitable for analyzing proliferation, viability, and cytotoxicity. The reduction of tetrazolium salts such as MTT and XTT to coloured formazan compounds or the bioreduction of resazurin only occurs in metabolically active cells. Actively proliferating cells increase their metabolic activity while cells exposed to toxins will have decreased activity.

Viability of cells can also be assessed by staining for markers of apoptosis (e.g. annexin V, caspases activators etc) or by assessing propidium iodide uptake as a sign of cell death. Cells that do not stain positive for such markers of apoptosis (e.g. AnnexinV, caspase activation) or that do not take up propidium iodide are viable cells.

In some embodiments, viability of cells is assessed by cell counting using viability dyes such as trypan blue. In preferred embodiments, more than 50%, more than 60%, more than 70%, more than 80%, more than 90%, more than 99% or all cells are viable after transduction, as assessed using trypan blue.

In some embodiments, viability of cells is assessed using flow cytometry. In preferred embodiments, more than 50%, more than 60%, more than 70%, more than 80%, more than 90%, more than 99% or all cells are viable after transduction, as assessed using flow cytometry.

There may be a recovery period prior to assessing cell viability. During the recovery period, the transduction buffer is removed from the cells and the cells are typically cultured in cell culture medium suitable for the particular cell type. In some embodiments, the recovery period is between 12 hours and 3 days.

In some embodiments, culture medium with post-iTOP viability enhancer (Human Serum at 5%) is added to iTOPed cells after iTOP. The inventors have found that this can enhance cell viability (see Example 13).

### Efficiency/Time for Transduction

The transduction buffers and methods of the invention allow improved transduction of proteins and nucleic acids into cells. The increased transduction efficiency of the buffers and methods of the invention means that high levels of transduction can be achieved after a shorter incubation time, thus representing a more convenient method of transduction. Transduction of proteins and nucleic acids into cells can be achieved significantly faster than with previously known methods and buffers.

In order to transduce a protein and/or nucleic acid into a cell, the protein and/or nucleic acid and cell are in contact for a sufficient length of time for the protein/nucleic acid to transduce into the cell. In general a cell or population of cells will be contacted with many copies of the protein and/or nucleic acid. Thus in a preferred embodiment, the protein(s) and/or nucleic acid(s) and cell(s) are in contact for a sufficient length of time for at least 40%, at least 50%, at least 60%, at least 70%, at least 80% or at least 90% of the protein(s) and/or nucleic acid(s) to transduce into the cell(s).

Generally, the amount of uptake into the cell correlates with the amount of time the cell is in contact with the transduction buffer and protein and/or nucleic acid. This is known herein as the “incubation time” or the “transduction time”.

In some embodiments, the incubation time is less than 90 minutes, less than 80 minutes, less than 70 minutes, less than 60 minutes, less than 50 minutes, less than 40 minutes, less than 30 minutes, less than 25 minutes, less than 20 minutes, less than 15 minutes, less than 10 minutes, less than 5 minutes, or less than 1 minute.

In a preferred embodiment, the incubation time is between 10 minutes and 45 minutes, most preferably between 15 minutes and 30 minutes. In some embodiments, the incubation time is about 15 minutes. In some embodiments, the incubation time is about 30 minutes. The efficiency of transduction during these preferred incubation periods is higher than using previously reported transduction buffers containing a transduction compound and a sodium-related salt.

In some embodiments of the invention, the transduction buffer or method allows more than 20%, more than 30%, more than 40%, more than 50%, more than 60%, more than 70%, more than 80%, more than 90%, or more than 95% knockout of a target gene, following a transduction time of less than 70 minutes, less than 60 minutes, less than 50 minutes, less than 40 minutes, less than 30 minutes, less than 20 minutes, less than 15 minutes, less than 10 minutes or less than 5 minutes.

The rate of transduction will depend upon the cell type (and the efficiency of transduction mechanisms) and the protein and/or nucleic acid to be transduced (its size, charge, hydrophobicity etc). For example, in some embodiments, induced pluripotent stem cells, preferably human induced pluripotent stem cells (hiPSCs), may be incubated with the transduction buffer for 10-20 minutes, preferably about 15 minutes. In some embodiments, T cells, preferably primary T cells, may be incubated with the transduction buffer for 10-30 minutes, preferably 10 minutes, 15 minutes, 20 minutes, or 30 minutes. In some embodiments, ARPE-19 cells may be incubated with the transduction buffer for 60 minutes. In some embodiments, Jurkat cells may be incubated with the transduction buffer for 45 minutes.

Transduction can be detected qualitatively or quantitatively using reporter constructs known in the art and available commercially, e.g. a luciferase or a GFP reporter construct, wherein levels of fluorescence correspond to levels of expression.

In some embodiments, transduction efficiency is assessed by measuring the level of knockout achieved in a target gene, for example when the transduced protein is part of a gene editing system or the transduced nucleic acid is a siRNA. Methods to measure the level of knockout of a target gene are known in the art, for example using flow cytometry (FACS), Inference of CRISPR Edits (ICE) or quantitative polymerase chain reaction (qPCR). In some preferred embodiments, knockout of a target gene is assessed using FACS.

In further preferred embodiments, knockout of a target gene is assessed using ICE. The Examples section provides further details of particularly preferred methods.

In some embodiments, the method comprises one round of transduction. However, in other embodiments, multiple rounds of transduction may be desirable. For example, in some embodiments 2, 3, 4, 5, 6, 7, 8, 9, 10 or more rounds of transduction are carried out on the same cells. Each round of transduction may involve transduction of the same protein and/or nucleic acid or of different proteins and/or nucleic acids. A particular advantage of the transduction buffers and methods of the invention is the ability to achieve high levels of transduction after only one round of transduction. Therefore, in a preferred embodiment, the method involves using only one round of transduction.

There may be a recovery period prior to assessing transduction efficiency. During the recovery period, the transduction buffer is removed from the cells and the cells are typically cultured in cell culture medium suitable for the particular cell type.

The Protein and/or Nucleic Acid for Transduction

The transduction buffer and methods of the invention can be used to transduce a protein, a nucleic acid, or a combination thereof into a cell.

In a preferred embodiment of the invention, the transduction buffer or method is suitable for transduction of a protein into a cell. In a further preferred embodiment, the transduction buffer or method is suitable for transduction of a protein and a nucleic acid into a cell, either simultaneously, sequentially or separately.

In some embodiments, the transduction buffer and methods can be used to transduce a protein. Non-limiting examples of proteins include monoclonal antibodies, cytokines, tissue growth factors and therapeutic proteins. In some embodiments, the protein is a biological drug (also known as a biologic).

The transduction methods disclosed herein are particularly useful for gene editing and gene therapy. For instance, the examples show that the transduction methods can be used to efficiently modify target genes following transduction of Cas9 proteins, and to efficiently knockdown gene expression following transduction of siRNA. Therefore, in some embodiments, the transduction method is a method for transducing a gene editing system into a cell. Gene editing systems comprise gene editing proteins and/or nucleic acids. For example, CRISPR/Cas systems typically comprise a Cas protein, which is a gene editing protein that is capable of modifying a nucleic acid, and a guide RNA. Proteins and nucleic acids used for DNA-editing and RNA-editing are well known in the art (East-Seletsky et al, Mol. Cell, 2017, 66(3) 373-383.e3; Makarova et al, Nat. Rev. Microbiol., 2015, 13(11) 722-736; O'Connell, 2019, J. Mol. Biol., 431(1), 66-87; Xu and Li, Comput Struct Biotechnol J., 2020, 18: 2401-2415). Therefore, in some embodiments, the protein and/or nucleic acid is part of a gene editing system.

As used herein, the term “gene editing protein” encompasses proteins capable of editing DNA, proteins capable of editing RNA, and proteins capable of modifying expression of genes e.g. by activating or inhibiting the transcription of target genes.

In some embodiments, the gene editing system comprises or consists of proteins that target a specific sequence, such as zinc finger nucleases (ZFNs) or TALENs. In some embodiments, the gene editing system comprises a protein that is guided to its target sequences by a (separate) guide molecule. Examples of such proteins that are guided to their target sequence include, but are not limited to, Cas proteins such as Cas9 nuclease, proteins from the Cascade system, TtAgo and other Argonaute proteins, and other FOKI-nuclease associated proteins. Therefore, in one embodiment, the transduction buffers and methods herein are used for the transduction of a gene editing system into cells selected from zinc finger nucleases, TALENS, Cas proteins, a Cascade complex, a TtAgo protein and an Argonaute protein.

In a preferred embodiment, the protein is a Cas protein. As used herein, the term “Cas protein” encompasses all CRISPR associated proteins, including but not limited to Cas9, Cas12 and Cas13. Examples of Cas9, Cas12 and Cas13 proteins include, but are not limited to, SpCas9, SaCas9, FnCas9, NmCas9, TCas9, Cas12a (Cpf1), Cas12b (C2c1), Cas12c (C2c3), Cas13a (C2c2), Cas13b (C2c4), C2c7, Cas13d and dCas9. Therefore, in one preferred embodiment, the gene editing protein is a Cas protein selected from Cas9, Cas12 and Cas13. In a further preferred embodiment, the gene editing protein is selected from SpCas9, SaCas9, FnCas9, NmCas9, TCas9, Cas12a (Cpf1), Cas12b (C2c1), Cas12c (C2c3), Cas13a (C2c2), Cas13b (C2c4), C2c7, Cas13d and dCas9. As explained below, any of these Cas proteins may be transduced into a cell in the form of a CRISPR-Cas system. Proteins for use in CRISPR-Cas systems are known in the art (see for example, Xu and Li, CRISPR-Cas systems: Overview, innovations and applications in human disease research and gene therapy. Comput Struct Biotechnol J., 2020, 18: 2401-2415).

More preferably, the protein is a Cas9. The examples demonstrate that the transduction buffers and methods of the invention are particularly useful for transducing Cas9 proteins into cells. As used herein, the term “Cas9” encompasses all variants of Cas9 isolated from different bacteria, including, but not limited to SpCas9, SaCas9, FnCas9, NmCas9 and TCas9. This term also encompasses modified versions of these variants, such as modified SpCas9. The term “modified SpCas9” refers to SpCas9 proteins in which amino acid sequences have been altered or added to the SpCas9 protein isolated from a bacterium. Preferably, a modified Cas9 has one or more nuclear localization sequence (NLS) added to it. The term “Cas9” therefore encompasses variants such as SpCas9 and modified versions of these variants such as SpCas9-1×NLS. The same definitions apply to Cas12 and Cas13.

Particularly preferred examples of Cas 9 proteins are SpCas9, most preferably SpCas9-1×NLS or 4×NLS-SpCas9-2×NLS.

Generally, Cas proteins are part of CRISPR-Cas gene editing systems. Any of the Cas proteins discussed above may be transduced in the form of a CRISPR-Cas system. Therefore, in one embodiment, the invention provides a method of transducing a CRISPR-Cas system into a cell, comprising contacting the cell with a transduction buffer disclosed herein and a CRISPR-Cas system.

In some embodiments, the protein is a part of a CRISPR-Cas system selected from CRISPR-Cas9, CRISPR-Prime, CRISPR-dCas9, CRISPR-Cas12, and CRISPR-Cas13. In a preferred embodiment, the CRISPR-Cas system is CRISPR-Cas9.

In some embodiments, the protein is transduced with a guide molecule. Preferably, the guide molecule is a guide nucleic acid, such as an sgRNA, a gDNA or a ctRNA. Guide nucleic acids, such as sgRNA, gDNA, or ctRNA can be designed by methods known in the art to target a specific sequence in the target nucleic acid (see for example, Mali, P., et al., RNA-guided human genome engineering via Cas9. Science, 2013, 339(6121): p. 823-6 for sgRNA; and Swarts, D. et al, DNA-guided DNA interference by a prokaryotic Argonaute. Nature, 2014, 507, 258-261 for gDNA). Thus, in some embodiments, the molecule for transduction is a guide nucleic acid, for example an sgRNA, a gDNA or a ctRNA.

A preferred example of a protein and nucleic acid combination which may be transduced using the transduction buffer and method of the invention is the combination of sgRNA and a Cas9 protein. Further specific preferred examples of protein and nucleic acid combinations include sgRNA and SpCas9; sgRNA and SpCas9-1×NLS; sgRNA and 4×NLS-SpCas9-2×NLS; ctRNA and SpCas9; ctRNA and SpCas9-1×NLS; and ctRNA and 4×NLS-SpCas9-2×NLS. In some embodiments, the nucleic acid and protein are present as nucleic acid-protein complexes.

In some embodiments, the protein has nuclease activity. In some embodiments, the protein lacks nuclease activity, for example the protein may be nuclease dead Cas9 (dCas9). The CRISPR-dCas9 system may be used to activate or inhibit the transcription of target genes (CRISPRa or CRISPRi) (see for example, Xu and Li, CRISPR-Cas systems: Overview, innovations and applications in human disease research and gene therapy. Comput Struct Biotechnol J., 2020, 18: 2401-2415). In some embodiments, the CRISPR-Cas system protein can edit RNA, for example Cas13 proteins such as Cas13a (C2c2), Cas13b, Cas13c and Cas13d. Such proteins can be used to transiently modify gene expression, for example in RNA knockdown.

Thus, in some embodiments, the transduction buffer and the methods are used to activate or inhibit the transcription of target genes, for example by introducing a protein and/or nucleic acid of the CRISPR-dCas9 system into the cell. The CRISPR-dCas9 system may be used to activate or inhibit the transcription of target genes.

In some embodiments, the transduction buffer and the methods can be used to edit or knockdown RNA, for example by introducing a protein and/or nucleic acid of the CRISPR-Cas system, for example Cas13 proteins such as Cas13a (C2c2), Cas13b, Cas13c and Cas13d (East-Seletsky et al, Mol. Cell, 2017, 66(3) 373-383.e3; Makarova et al, Nat. Rev. Microbiol., 2015, 13(11) 722-736; O'Connell, 2019, J. Mol. Biol., 431(1), 66-87; Xu and Li, Comput Struct Biotechnol J., 2020, 18: 2401-2415). Such proteins can transiently modify gene expression, for example in RNA knockdown.

In some embodiments, the transduction buffer and methods can be used to transduce a nucleic acid. In some embodiments, the nucleic acid is DNA, cDNA, RNA, miRNA, siRNA or any modified version thereof. In some embodiments, the nucleic acid is small guide RNA (sgRNA), for example, for use with the CRISPR-Cas9 gene editing system or other gene editing systems, or a small guide DNA (gDNA), for example, for use with the TtAgo gene editing system or other gene editing systems. Therefore, in some embodiments, the nucleic acid is a guide nucleic acid (such as an sgRNA, a gDNA or a ctRNA). In preferred embodiments, when the method of the invention is used to transduce a nucleic acid into a cell, the nucleic acid is an sgRNA or ctRNA for use with the CRISPR-Cas9 gene editing system.

In some embodiments, the nucleic acid is an RNA for use in RNA interference (for example, an siRNA). RNA interference can transiently knockdown expression of a target gene, by selectively targeting mRNA for destruction. Thus, in some preferred embodiments, the nucleic acid is an siRNA.

In some embodiments, the transduction buffer and the methods of the invention can be used to knockdown gene expression via RNA interference, for example by introducing a siRNA into the cell. siRNAs can selectively target mRNA for destruction, resulting in a temporary reduction in expression of the target gene.

In some embodiments, more than one protein and/or nucleic acid (i.e. multiple copies of the protein and/or nucleic acid) is transduced into a cell. For example, at least 2, at least 10, at least 20, at least 50, at least 100, at least 500, at least 1000, at least 2000, at least 5000, at least 10,000 molecules of interest, at least 10⁴, at least 10⁵, at least 10⁶, at least 10⁷, or more than 10⁷ proteins and/or nucleic acids are transduced into the cell.

In some embodiments, one or more (e.g. 1, 2, 3, 4, 5, 6, 7, 8, 9, 10 or more) different types of proteins and/or nucleic acids are transduced into a cell. In some embodiments, multiple proteins and/or nucleic acids are transduced into the cell, for example one or more proteins and one or more nucleic acids.

In some embodiments, the protein or nucleic acid is between about 30 kDa to about 500 kDa, for example between about 30 kDa and about 200 kDa. For example, in some embodiments, the protein or nucleic acid is about 30, about 40, about 50, about 60, about 70, about 80, about 90, about 100, about 110, about 120, about 130, about 140, about 150, about 160, about 170, about 180, about 190, or about 200 kDa. In some embodiments, the protein or nucleic acid is more than 30, more than 40, more than 50, more than 60, more than 70, more than 80, more than 90, more than 100, more than 110, more than 120, more than 130, more than 140, more than 150, more than 160, more than 170, more than 180, more than 190, or more than 200 kDa. These sizes are particularly applicable where the molecule for transduction is a protein. Where the molecule for transduction is a nucleic acid molecule, such as an oligonucleotide or a polynucleotide, the size is typically defined by the number of nucleotides.

In some embodiments, the transduction buffer comprises one protein or nucleic acid for transduction. In other embodiments, the transduction buffer comprises more than one protein and/or nucleic acid for transduction, for example two, three, four, five or more proteins and/or nucleic acids.

The same principle applies for the methods of the invention, i.e. the cell may be contacted with two, three, four, five or more proteins and/or nucleic acids. In some embodiments, the cell may be contacted with a protein and a nucleic acid. For example, an SpCas9 protein and a nucleic acid may be transduced into a cell simultaneously.

The concentration of the protein and/or nucleic acid for transduction depends upon the protein/nucleic acid, the cell, and the purpose of transduction. A particular advantage of the improved buffers and methods of the invention is the ability to achieve efficient transduction at a significantly lower concentration of protein and/or nucleic acid than using previously reported buffers and methods. In particular, in some embodiments, it is possible to use 5× lower concentration of protein and/or nucleic acid to achieve the same level of transduction.

The skilled person can determine the appropriate concentration. In some embodiments, the protein and/or nucleic acid for transduction is added at millimolar, micromolar or nanomolar concentrations, preferably at micromolar concentrations. In some embodiments, the protein and/or nucleic acid is added to the transduction buffer at a concentration of between about 0.5 μM and between about 40 μM, between about 0.5 M and between about 20 μM or between about 0.5 μM and 15 μM. In some embodiments, the concentration of the protein and/or nucleic acid is about 5 μM. In some embodiments, the concentration of the protein and/or nucleic acid is about 10 μM. In some embodiments, the concentration of the protein and/or nucleic acid is about 20 μM. Preferably, the protein and/or nucleic acid is added to the transduction buffer at a concentration of between about 0.5 μM and about 15 μm.

In some embodiments, a Cas protein, preferably a Cas9 protein, is added to the transduction buffer at a concentration of between about 0.5 μM and between about 40 μM, between about 0.5 μM and between about 20 μM or between about 0.5 μM and 15 μM. In some embodiments, the concentration of the Cas protein, preferably a Cas9 protein, is about 5 μM. In some embodiments, the concentration of the Cas protein, preferably a Cas9 protein, is about 10 μM. In some embodiments, the concentration of the Cas protein, preferably a Cas9 protein, is about 20 μM. Preferably, the Cas protein, preferably a Cas9 protein, is added to the transduction buffer at a concentration of between about 0.5 μM and about 15 μm.

### Cell for Transduction

The transduction method can be used to transduce a protein and/or nucleic acid into any cell.

In a preferred embodiment, the cell involved in the transduction method is a mammalian cell, such as a human, primate, rodent (e.g. mouse or rat), rabbit, dog, cat, horse, cow or pig cell. These mammals are useful for research purposes and/or may benefit from treatment or diagnosis comprising transduction buffers and methods of the invention. Among these cell types, human cells are most preferred for use with the transduction buffers and methods of the invention.

In some embodiments the cell is in vivo, optionally in situ. For example, when treating or diagnosing a medical condition, the protein and/or nucleic acid could be administered directly and locally in combination with the transduction buffer to an organism or tissue in need thereof.

In some embodiments, the cell is in vitro. For example, the cell may be in a culture medium, wherein the culture medium optionally supports the maintenance, differentiation and/or expansion of the cell.

In some embodiments, the cell is derived from an established cell line, such as an established human cell line. In some embodiments, the established cell line is an immortalised cell line. In other embodiments the cell line is a primary cell line.

Examples of established human cell lines suitable for use in the context of the invention include but are not limited to ARPE-19 (human retinal epithelial cell line), Jurkat (human T lymphocyte cell line), HEK293 (human embryonic kidney cell line), HeLa, ESTDAB database, DU145 (prostate cancer), Lncap (prostate cancer), MCF-7 (breast cancer), MDA-MB-438 (breast cancer), PC3 (prostate cancer), T47D (breast cancer), THP-1 (acute myeloid leukemia), COS7 (immortalised CV-1 cells from kidney tissue), U87 (glioblastoma), SHSY5Y human neuroblastoma cells, cloned from a myeloma, Saos-2 cells (bone cancer). The ESTDAB database (www.ebi.ac.uk/ipd/estdab/directory.html) and National Cancer Institute (NCI-60) provide further examples of cancer cell lines which are suitable for use with the present invention. In some embodiments, the established cell line is a primate cell line, such as Vero (African green monkey Chlorocebus kidney epithelial cell line initiated in 1962). In some embodiments, the established cell line is a rodent cell line, such as GH3 (pituitary tumor), PC12 (pheochromocytoma) or MC3T3 (embryonic calvarium). Other mammalian cell lines suitable for use with the transduction buffer and methods disclosed herein include the Madin-Darby canine kidney (MDCK) epithelial cell line, Chinese hamster ovary (CHO) cell line and Caco-2 cells. In some embodiments the cell is a KBM7 cell.

The transduction buffer of the invention has been shown to be particularly effective for transducing proteins and nucleic acids into ARPE-19 cells, HEK293 cells, Jurkat cells, T cells and induced pluripotent stem cells. Therefore, in some preferred embodiments, the cell is an ARPE-19 cell, HEK293 cell, Jurkat cell, T cell or induced pluripotent stem cell.

It is known in the art that transduction can be difficult in in some cell types, such as human induced pluripotent stem cells (hiPSCs) and primary T cells. It has surprisingly been found that the transduction buffers and methods of the invention can be used to transduce proteins and nucleic acids into difficult-to-transduce cell lines, such as hiPSCs and primary T cells. In some embodiments, the cell is a difficult-to-transduce cell. Preferably, the cell is an induced pluripotent stem cell or a T cell. In particularly preferred embodiments, the cell is a hiPSC or a primary T cell.

As explained above, a particular advantage of the transduction buffers and methods of the invention is the ability to transduce proteins that are challenging to transduce into cells, such as Cas9 proteins, into difficult-to-transduce cells. Thus, in one preferred embodiment, the invention provides a method for transducing a Cas9 protein into an induced pluripotent stem cell or a T cell, preferably a hiPSC or a primary T cell. comprising contacting said cell with a transduction buffer disclosed herein and a Cas9 protein. In a further preferred embodiment, the invention provides a method for transducing spCas9-1×NLS or 4×NLS-spCas9-2×NLS into an induced pluripotent stem cell or a T cell, preferably a hiPSC or a primary T cell, comprising contacting said cell with a transduction buffer disclosed herein and spCas9-1×NLS or 4×NLS-spCas9-2×NLS.

In some embodiments, the cell is a primary cell. A primary cell or cell line is derived from a cell taken directly from a living organism, and has not been immortalized. In other words, a primary cell or cell line is genetically and phenotypically stable. In some particularly preferred embodiments, the cell is a primary T cell, such as a CD4+ T cell.

In some embodiments, the cell is a stem cell or a cell derived by differentiation of a stem cell. In some embodiments the stem cell is a pluripotent stem cell, such as an embryonic stem cell, optionally a human embryonic stem cell. In some embodiments, the cell is not a human embryonic stem cell. In some embodiments, the stem cell is not obtained by methods that involve the use of human embryos for commercial or industrial purposes. In some embodiments, the stem cell is not obtained by methods that necessarily involve the destruction of a human embryo. In some embodiments the stem cell is a murine embryonic stem cell. In other embodiments, the stem cell is an adult stem cell, such as a neural, adipose or hematopoietic stem cell. In some embodiments, the cell is a murine or human neural stem cell, neuron cell or glia cell. In some embodiments, the cell is a somatic cell or a germ cell. In some preferred embodiments, the stem cell is an induced pluripotent stem cell. In some particularly preferred embodiments, the cells is a human induced pluripotent stem cell (hiPSC).

In some embodiments, the cell is a cell belonging to the immune system, such as a T cell, B cell or leukocyte, including but not limited to a phagocyte (macrophage, neutrophil, or dendritic cell), mast cell, eosinophil, basophil, and natural killer cell. In some preferred embodiments, the cell is a T cell, particularly preferably a primary T cell. In some embodiments, the cell is a CD4+ T cell. In some embodiments, the cell is a dendritic cell.

In some embodiments, the cell is a suspension cell, including for example a T cell or a Jurkat cell. In some particularly preferred embodiments, the cell is a primary T cell, such as a CD4+ cell. In some embodiments, the cell is a Jurkat cell. In some embodiments, the cell is an adherent cell. In some embodiments, the cell is an ARPE-19 cell.

In some embodiments, the cells for transduction are cultured in an atmosphere comprising between about 4% and about 10% CO₂, about 5% and about 9% CO₂, about 6% and about 8% CO₂, preferably about 5% CO₂.

In all embodiments, where the disclosure refers to a “cell”, it refers to a single cell and also applies to a “cell population”, for example of 2 or more, 10 or more, 100 or more, 1000 or more, 10⁴ or more, 10⁵ or more, 10⁶ or more, 10⁷ or more, 10⁸ or more cells.

### Pharmaceutical Composition

The invention provides a pharmaceutical composition comprising the transduction buffer and a pharmaceutically acceptable carrier. In some embodiments, invention provides a pharmaceutical composition comprising the transduction buffer of the invention, a pharmaceutically acceptable carrier and a protein or nucleic acid, or a combination thereof. In some embodiments, the protein or nucleic acid, or a combination thereof, and transduction buffer components are administered simultaneously or sequentially.

The pharmaceutical composition can include further components in addition to the transduction buffer and protein or nucleic acid, or a combination thereof. The pharmaceutical composition will typically include a pharmaceutically acceptable carrier, which can be any substance that does not itself induce the production of antibodies harmful to the patient receiving the composition, and which can be administered without undue toxicity. Suitable pharmaceutically acceptable carriers are well known in the art. Pharmaceutically acceptable carriers can, for example, include liquids such as water, saline, glycerol and ethanol. Auxiliary substances, such as wetting or emulsifying agents, pH buffering substances, and the like, can also be present in pharmaceutical compositions (see Gennaro (2000) Remington: The Science and Practice of Pharmacy. 20th edition, ISBN: 0683306472).

In some embodiments, there is provided a pharmaceutical composition comprising a transduction compound or transduction compound and a gene editing protein, such as a gene editing protein that is part of a gene editing system.

The pharmaceutical composition may be sterile and/or pyrogen-free.

The invention also provides a container (e.g. vial) or delivery device (e.g. syringe) pre-filled with a pharmaceutical composition of the invention. The invention also provides a process for providing such a container or device, comprising introducing into the container or device a composition of the invention.

The appropriate dose may vary depending upon the health and physical condition of the individual to be treated, age, the taxonomic group of individual to be treated (e.g. human, non-human primate, primate, etc.), the degree of transduction desired, the formulation of the pharmaceutical composition, the treating doctor's assessment of the medical situation, and other relevant factors. The dose may fall in a relatively broad range that can be determined through routine trials.

Compositions of the invention may be prepared in various liquid forms. For example, the compositions may be prepared as injectables, either as solutions or suspensions. Injectables for local sub-cutaneous or intramuscular administration are typical. Injection may be via a needle (e.g. a hypodermic needle), but needle-free injection may alternatively be used.

Compositions may include an antimicrobial. Antimicrobials such as thiomersal and 2-phenoxyethanol are commonly found in pharmaceutical compositions, but it is preferred to use either a mercury-free preservative or no preservative at all.

Compositions may comprise detergent e.g. a Tween (polysorbate), such as Tween 80. Detergents are generally present at low levels e.g. <0.01%. In some embodiments, the buffer does not comprise a detergent.

In some embodiments, the method for transduction does not involve the use of a detergent during transduction.

Effective dosage volumes can be routinely established, depending on the purpose of the composition. Typical human dose of the composition might be, for example about 0.5 ml e.g. for intramuscular injection (e.g. local injection into the muscle or tissue of interest). Similar doses may be used for other delivery routes.

The invention also provides a kit comprising a transduction buffer of the invention or a pharmaceutical composition of the invention. The kit may additionally comprise cells and/or the protein, nucleic acid or combination thereof for transduction. The kit may also comprise instructions for use. The kit may include the various components of the transduction buffer in one or more separate containers, e.g. 1, 2, 3, 4, 5, 6 or more separate containers. For example, the kit may comprise a container comprising a salt solution, a container comprising the transduction compound, a container comprising the protein, nucleic acid or combination thereof, a container comprising the osmoprotectant and/or a container comprising a diluent or media. In addition the kit may comprise any one or more of the additional other components as described herein, wherein they are suitable for simultaneous, sequential or separate administration with the transduction buffer.

### Therapeutic Uses of the Invention

The invention provides a transduction buffer as disclosed herein or a pharmaceutical composition comprising said buffer and a pharmaceutically acceptable carrier, for use in therapy.

In a preferred embodiment, the method of therapy comprises contacting cells with a transduction buffer or pharmaceutical composition disclosed herein and a gene editing protein selected from zinc finger nucleases. TALENS, Cas proteins (preferably Cas9, Cas12 or Cas13), a Cascade complex, a TtAgo protein and an Argonaute protein, or a CRISPR-Cas system selected from CRISPR-Cas9, CRISPR-Prime, CRISPR-dCas9, CRISPR-Cas12, and CRISPR-Cas13. In a particularly preferable embodiment, the gene editing protein is a Cas9 protein.

In one embodiment, the gene editing protein is selected from SpCas9, SaCas9, FnCas9, NmCas9, TCas9, Cas12a (Cpf1), Cas12b (C2c1), Cas12c (C2c3), Cas13a (C2c2), Cas13b (C2c4), C2c7, Cas13d and dCas9.

As explained above, the transduction buffers and methods of the present invention are particularly useful for transducing proteins capable of modifying a nucleic acid, in particular difficult-to-transduce gene editing proteins such as Cas9 proteins, into cells. In some embodiments, the therapy is gene therapy, e.g. for treatment of genetic disorders, including inherited disorders. The present invention thereof provides the transduction buffers disclosed herein for use in treating a genetic disorder. Genetic disorders that can be treated in accordance with the invention include, but are not limited to the following common disorders: 22q11.2 deletion syndrome, Angelman syndrome, Canavan disease, Charcot-Marie-Tooth disease, Color blindness, Cri du chat, Cystic fibrosis, Down syndrome, Haemochromatosis, Haemophilia, Klinefelter syndrome, Neurofibromatosis, Phenylketonuria, Polycystic kidney disease, Prader-Willi syndrome, Sickle-cell disease, Tay-Sachs disease, Turner syndrome. In a preferred embodiment, the treatment of genetic disorders, including the disorders listed above, comprises contacting cells with a transduction buffer or pharmaceutical composition disclosed herein and a gene editing protein selected from zinc finger nucleases, TALENS, Cas proteins, a Cascade complex, a TtAgo protein and an Argonaute protein, or a CRISPR-Cas system selected from CRISPR-Cas9, CRISPR-Prime, CRISPR-dCas9, CRISPR-Cas12, and CRISPR-Cas13. In a particularly preferred embodiment, the gene editing protein is a Cas protein, most preferably Cas9.

The invention also provides a method of treating a genetic disorder by contacting a cell with a transduction buffer disclosed herein and a gene editing protein, thereby transducing the gene editing protein into the cell. In the method of treating a genetic disorder, the genetic disorder and the gene editing protein may be as defined as immediately above.

The invention also provides a method of treating a genetic disease in a subject, by contacting a cell with a transduction buffer or pharmaceutical composition disclosed herein and a gene editing protein, wherein the gene editing protein is transduced into the cell, and wherein the genetic disease is treated.

In some embodiments, the method can be used to modify genes that pathogens use to cause disease, and thus can be used to treat infection. Therefore in some embodiments, the therapy is for treatment of infectious diseases, for example including but not limited to HIV, malaria. African Trypanosomiasis. Cholera. Cryptosporidiosis. Dengue. Hepatitis A/B/C. Influenza. Japanese Encephalitis, Leishmaniasis. Measles. Meningitis. Onchocerciasis (“river blindness”). Pneumonia. Rotavirus, Schistosomiasis. Shigellosis. Strep Throat. Tuberculosis. Typhoid. Typhoid. In a preferred embodiment, the treatment of infectious diseases. including the infectious diseases listed above, comprises contacting cells with a transduction buffer or pharmaceutical composition disclosed herein and a gene editing protein selected from zinc finger nucleases. TALENS. Cas proteins, a Cascade complex, a TtAgo protein and an Argonaute protein, or a CRISPR-Cas system selected from CRISPR-Cas9. CRISPR-Prime. CRISPR-dCas9. CRISPR-Cas12, and CRISPR-Cas13. In a particularly preferred embodiment, the gene editing protein is a Cas protein, most preferably Cas9.

The invention also provides a method of treating an infectious disease by contacting a cell with a transduction buffer disclosed herein and a gene editing protein, thereby transducing the gene protein into the cell. In the method of treating an infectious disease, the infectious disease and the gene editing protein may be as defined in immediately above.

In some embodiments, the invention provides a transduction buffer or pharmaceutical composition as disclosed herein, for use in a method of treating or preventing a disease or condition selected from the group consisting of: HIV. Hemophilia B. Mucopolysaccharidosis I. Mucopolysaccharidosis II. Sickle Cell Disease. Thalassemia (such as β-Thalassemia. Thalassemia Major. β-thalassemia Major, and Transfusion Dependent Beta-thalassemia), Leber Congenital Amaurosis 10 (LAC10). Human Papillomavirus-Related Malignant Neoplasm. Acute Myeloid Leukemia. Multiple Myeloma. B-cell Acute Lymphoblastic Leukemia. Metastatic Non-small Cell Lung Cancer. B Cell Leukemia/B Cell Lymphoma. EBV positive advanced stage malignancies. Esophageal Cancer. T cell malignancy. Solid Tumor. Melanoma. Synovial Sarcoma. Liposarcoma. B-cell malignancies. D19⁺ leukemia or lymphoma. Gastro-Intestinal (GI) Cancer. Renal Cell Carcinoma, and Advanced Hepatocellular Carcinoma. These disorders are all the subject of clinical trials of gene therapy using genome-editing technology (see Xu and Li. CRISPR-Cas systems: Overview, innovations and applications in human disease research and gene therapy. Comput Struct Biotechnol J., 2020, 18: 2401-2415). In a preferred embodiment, the treatment of the above diseases comprises contacting cells with a transduction buffer or pharmaceutical composition disclosed herein and a gene editing protein selected from zinc finger nucleases, TALENS, Cas proteins, a Cascade complex, a TtAgo protein and an Argonaute protein, or a CRISPR-Cas system selected from CRISPR-Cas9, CRISPR-Prime, CRISPR-dCas9, CRISPR-Cas12, and CRISPR-Cas13. In a particularly preferred embodiment, the gene editing protein is a Cas protein, most preferably Cas9.

The invention also provides a method of treating a disease listed above by contacting a cell with a transduction buffer disclosed herein and a gene editing protein, thereby transducing the gene protein into the cell. In the method of treating such a disease, the gene editing protein may be as defined in immediately above.

The invention also provides methods for therapy or diagnosis comprising transducing a protein and/or nucleic acid into a cell. The cell may be an in vivo cell, in which case the treatment is a direct treatment. Alternatively, the cell may be transduced in vitro, e.g. for in vitro diagnosis. Alternatively, the cell may be transduced in vitro prior to transplantation of the cell into a patient. For example, the transduction buffer or methods of the invention may be used to introduce a chimeric antigen receptor to T cells, for use in CAR-T cell therapy. The transduction buffer and methods of the invention may be particularly suitable for this application, due to their ability to efficiently transduce primary T cells. The transplantation of the cell into the patient may be autologous or allogenic, i.e. the transduced cell may be transplanted back into the same patient that it was taken from (autologous) or into a different person (allogenic). In a preferred embodiment the transplantation is autologous.

Biological drugs (also known as biologics) including monoclonal antibodies, cytokines, tissue growth factors and therapeutic proteins are becoming increasingly important alternatives to chemical small molecules for use in therapy. However, there are a number of difficulties associated with biological drugs, in particular relating to their delivery to the target of interest. The transduction buffers and methods of the present invention could be used to improve delivery of biologics to cells. For example, in some embodiments, the protein in the methods of the invention is a biologic, for example selected from a monoclonal antibody, cytokine, tissue growth factor and therapeutic protein. The cell of interest may be transduced in vitro and transplanted back into the patient, or the cell may be transduced in vivo.

In some embodiments, there is provided a gene editing protein or a gene editing system (such as CRISPR-Cas9, CRISPR-Prime, CRISPR-Cas12, ZNF, TALEN, the Cascade system, TtAgo and other Argonaute systems), for use in therapy. In some embodiments, said therapy comprises a method of transducing a protein and/or nucleic acid into a cell according to the invention. A number of diseases and conditions can be treated by transduction of proteins that modify a nucleic acid, for example gene editing systems, and it would be clear to the skilled person which diseases or conditions can be treated. Conditions and diseases treatable by transduction of a protein that modifies a nucleic acid, for example a gene editing system, include but are not limited to genetic diseases such as, sickle cell disease, Leber's congenital amaurosis, X-linked SCID, ADA-SCID, adrenoleukodystrophy, chronic lymphocytic leukemia (CLL), acute lymphocytic leukemia (ALL), multiple myeloma, haemophilia and Parkinson's disease. The therapy may be somatic or germline gene therapy, i.e. in some embodiments, the cell in the transduction method is a somatic cell or a germ cell.

### Exemplary Transduction Buffers and Methods

Non-limiting examples of transduction buffers and methods are provided below. It is to be understood that any combination of compatible embodiments described herein can be used for a transduction buffer or method for transduction comprising a transduction buffer. Some examples of combinable embodiments are provided below.

In some embodiments, the present invention provides a method for transducing a protein, nucleic acid or a combination thereof into a cell, wherein the method comprises contacting said cell with the protein or nucleic acid and a transduction buffer, wherein the transduction buffer comprises:
(i) a transduction compound selected from GABA or NDSB-201, preferably GABA;
(ii) a salt selected from a sodium, rubidium, lithium, potassium or caesium salt, wherein the total concentration of the sodium, rubidium, lithium, potassium or caesium salt is between 500 mM and 1100 mM;
(ii) one or more amino acids selected from serine, asparagine, cysteine, glutamine and threonine, and combinations thereof, wherein the total concentration of amino acids is between 75 mM and 250 mM; and
(iv) one or more disaccharides, wherein the total concentration of disaccharides is between 20 mM and 80 mM.

In some embodiments, the present invention provides the transduction buffer described above.

In some embodiments of the method and transduction buffer described above, the transduction buffer further comprises glycerol at a concentration of between 1000 mM and 2000 mM, preferably between 1300 mM and 1700 mM.

In some embodiments, the method described above is performed in vitro.

In some embodiments, the present invention provides a method for transducing a protein, nucleic acid or a combination thereof into a cell, wherein the method comprises contacting said cell with the protein or nucleic acid and a transduction buffer, wherein the transduction buffer comprises:
(i) a transduction compound selected from GABA or NDSB-201, preferably GABA;
(ii) a salt selected from a sodium, rubidium, lithium, potassium or caesium salt, wherein the total concentration of the sodium, rubidium, lithium, potassium or caesium salt is between 750 mM and 850 mM;
(ii) one or more amino acids selected from serine, asparagine, cysteine, glutamine and threonine, and combinations thereof, wherein the total concentration of amino acids is between 125 mM and 175 mM; and
(iv) one or more disaccharides, wherein the total concentration of disaccharides is between 40 mM and 60 mM.

In some embodiments, the present invention provides the transduction buffer described above.

In some embodiments of the method and transduction buffer described above, the transduction buffer further comprises glycerol at a concentration of between 1000 mM and 2000 mM, preferably between 1300 mM and 1700 mM.

In some embodiments, the method described above is performed in vitro.

In some embodiments, the present invention provides a method for transducing a protein, nucleic acid or a combination thereof into a cell, wherein the method comprises contacting said cell with the protein or nucleic acid and a transduction buffer, wherein the transduction buffer comprises:
(i) a transduction compound selected from GABA or NDSB-201, preferably GABA;
(ii) a salt selected from a sodium, rubidium, lithium, potassium or caesium salt, wherein the total concentration of the sodium, rubidium, lithium, potassium or caesium salt is between 500 mM and 1100 mM;
(ii) serine, wherein the total concentration of serine is between 75 mM and 250 mM; and
(iv) trehalose, wherein the total concentration of trehalose is between 20 mM and 80 mM.

In some embodiments, the present invention provides the transduction buffer described above.

In some embodiments of the method and transduction buffer described above, the transduction buffer further comprises glycerol at a concentration of between 1000 mM and 2000 mM, preferably between 1300 mM and 1700 mM.

In some embodiments, the method described above is performed in vitro.

In some embodiments, the present invention provides a method for transducing a protein, nucleic acid or a combination thereof into a cell, wherein the method comprises contacting said cell with the protein or nucleic acid and a transduction buffer, wherein the transduction buffer comprises:
(i) a transduction compound selected from GABA or NDSB-201, preferably GABA;
(ii) a salt selected from a sodium, rubidium, lithium, potassium or caesium salt, wherein the total concentration of the sodium, rubidium, lithium, potassium or caesium salt is between 750 mM and 850 mM;
(ii) serine, wherein the total concentration of serine is between 125 mM and 175 mM; and
(iv) trehalose, wherein the total concentration of trehalose is between 40 mM and 60 mM.

In some embodiments, the present invention provides the transduction buffer described above.

In some embodiments of the method and transduction buffer described above, the transduction buffer further comprises glycerol at a concentration of between 1000 mM and 2000 mM, preferably between 1300 mM and 1700 mM.

In some embodiments, the method described above is performed in vitro.

In some embodiments, the present invention provides a method for transducing a Cas protein, preferably Cas9, into a cell, wherein the method comprises contacting said cell with the protein or nucleic acid and a transduction buffer, wherein the transduction buffer comprises:
(i) a transduction compound selected from GABA or NDSB-201, preferably GABA;
(ii) a salt selected from a sodium, rubidium, lithium, potassium or caesium salt, wherein the total concentration of the sodium, rubidium, lithium, potassium or caesium salt is between 750 mM and 850 mM;
(ii) one or more amino acids selected from serine, asparagine, cysteine, glutamine and threonine, and combinations thereof, wherein the total concentration of amino acids is between 125 mM and 175 mM; and
(iv) one or more disaccharides, wherein the total concentration of disaccharides is between 40 mM and 60 mM.

In some embodiments of the method described above, the transduction buffer further comprises glycerol at a concentration of between 1000 mM and 2000 mM, preferably between 1300 mM and 1700 mM.

In some embodiments, the method described above is performed in vitro.

In some embodiments, the present invention provides a method for transducing a Cas protein, preferably Cas9, into a cell, wherein the method comprises contacting said cell with the protein or nucleic acid and a transduction buffer, wherein the transduction buffer comprises:
(i) a transduction compound selected from GABA or NDSB-201, preferably GABA;
(ii) a salt selected from a sodium, rubidium, lithium, potassium or caesium salt, wherein the total concentration of the sodium, rubidium, lithium, potassium or caesium salt is between 500 mM and 1100 mM;
(ii) serine, wherein the total concentration of serine is between 75 mM and 250 mM; and
(iv) trehalose, wherein the total concentration of trehalose is between 20 mM and 80 mM.

In some embodiments of the method described above, the transduction buffer further comprises glycerol at a concentration of between 1000 mM and 2000 mM, preferably between 1300 mM and 1700 mM.

In some embodiments, the method described above is performed in vitro.

In some embodiments, the present invention provides a method for transducing a protein, nucleic acid or a combination thereof into an induced pluripotent stem cell or a T cell, preferably a human induced pluripotent stem cell or a primary T cell, wherein the method comprises contacting said cell with the protein or nucleic acid and a transduction buffer, wherein the transduction buffer comprises:
(i) a transduction compound selected from GABA or NDSB-201, preferably GABA;
(ii) a salt selected from a sodium, rubidium, lithium, potassium or caesium salt, wherein the total concentration of the sodium, rubidium, lithium, potassium or caesium salt is between 750 mM and 850 mM;
(ii) one or more amino acids selected from serine, asparagine, cysteine, glutamine and threonine, and combinations thereof, wherein the total concentration of amino acids is between 125 mM and 175 mM; and
(iv) one or more disaccharides, wherein the total concentration of disaccharides is between 40 mM and 60 mM.

In some embodiments of the method described above, the transduction buffer further comprises glycerol at a concentration of between 1000 mM and 2000 mM, preferably between 1300 mM and 1700 mM.

In some embodiments, the method described above is performed in vitro.

In some embodiments, the present invention provides a method for transducing a protein, nucleic acid or a combination thereof into an induced pluripotent stem cell or a T cell, preferably a human induced pluripotent stem cell or a primary T cell, wherein the method comprises contacting said cell with the protein or nucleic acid and a transduction buffer, wherein the transduction buffer comprises:
(i) a transduction compound selected from GABA or NDSB-201, preferably GABA;
(ii) a salt selected from a sodium, rubidium, lithium, potassium or caesium salt, wherein the total concentration of the sodium, rubidium, lithium, potassium or caesium salt is between 500 mM and 1100 mM;
(ii) serine, wherein the total concentration of serine is between 75 mM and 250 mM; and
(iv) trehalose, wherein the total concentration of trehalose is between 20 mM and 80 mM.

In some embodiments of the method described above, the transduction buffer further comprises glycerol at a concentration of between 1000 mM and 2000 mM, preferably between 1300 mM and 1700 mM.

In some embodiments, the method described above is performed in vitro.

In some embodiments, the present invention provides a method for transducing a Cas protein, preferably Cas9, into an induced pluripotent stem cell or a T cell, preferably a human induced pluripotent stem cell or a primary T cell, wherein the method comprises contacting said cell with the protein or nucleic acid and a transduction buffer, wherein the transduction buffer comprises:
(i) a transduction compound selected from GABA or NDSB-201, preferably GABA;
(ii) a salt selected from a sodium, rubidium, lithium, potassium or caesium salt, wherein the total concentration of the sodium, rubidium, lithium, potassium or caesium salt is between 750 mM and 850 mM;
(ii) one or more amino acids selected from serine, asparagine, cysteine, glutamine and threonine, and combinations thereof, wherein the total concentration of amino acids is between 125 mM and 175 mM; and
(iv) one or more disaccharides, wherein the total concentration of disaccharides is between 40 mM and 60 mM.

In some embodiments of the method described above, the transduction buffer further comprises glycerol at a concentration of between 1000 mM and 2000 mM, preferably between 1300 mM and 1700 mM.

In some embodiments, the method described above is performed in vitro.

In some embodiments, the present invention provides a method for transducing a Cas protein, preferably Cas9, into an induced pluripotent stem cell or a T cell, preferably a human induced pluripotent stem cell or a primary T cell, wherein the method comprises contacting said cell with the protein or nucleic acid and a transduction buffer, wherein the transduction buffer comprises:
(i) a transduction compound selected from GABA or NDSB-201, preferably GABA;
(ii) a salt selected from a sodium, rubidium, lithium, potassium or caesium salt, wherein the total concentration of the sodium, rubidium, lithium, potassium or caesium salt is between 500 mM and 1100 mM;
(ii) serine, wherein the total concentration of serine is between 75 mM and 250 mM; and
(iv) trehalose, wherein the total concentration of trehalose is between 20 mM and 80 mM.

In some embodiments of the method described above, the transduction buffer further comprises glycerol at a concentration of between 1000 mM and 2000 mM, preferably between 1300 mM and 1700 mM.

In some embodiments, the method described above is performed in vitro.

### GENERAL

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x±10%. It also refers specifically to the exact value, e.g in the above example, to exactly 10%. Where necessary, the word “about” may be omitted from the definition of the invention.

The term “a” or “an”, unless specifically stated otherwise, means “one or more”. For example, it can mean “only one” or it can mean “more than one”, for example “two, three, four, five or more”.

Unless specifically stated, a process comprising a step of mixing two or more components does not require any specific order of mixing. Thus components can be mixed in any order. Where there are three components then two components can be combined with each other, and then the combination may be combined with the third component, etc.

It will be understood that the invention will be described by way of example only and modifications may be made whilst remaining within the scope and spirit of the invention.

### BRIEF DESCRIPTION OF DRAWINGS

FIG. 1: Improved transduction efficiency in hiPSCs using NT2100.2

Histogram showing knockout (KO) efficiency in hiPSCs following incubation with control (SB) or NT2100.2 buffer. Human induced pluripotent stem cells (hIPSCs) were contacted with SB (control) or NT2100.2 transduction buffer, spCas9-1×NLS and a sgRNA targeting B2M. Cells were incubated with transduction buffer, SpCas9-1×NLS and sgRNA for 15 min (NT2100.2; SB) or 45 min (SB). Total percentage of B2M knockout (KO) cells is shown in the histogram plot.

FIG. 2: Improved knockout efficiency in hIPSCs using NT2100.2 Histogram showing knockout (KO) efficiency in hiPSCs following incubation with control (SB) or NT2100.2 buffer. The assay described in Example 2 was repeated, with incubation periods of 15 min (NT2100.2 (n=2) and SB (n=2)) or 30 min (SB). Total percentage of B2M knockout (KO) cells is shown in the histogram plot.

FIG. 3: Improved knockout efficiency in primary T cells using NT2100 and NT2100.2

Primary T cells were incubated with NT2100, NT2100.2, or SB (control) transduction buffer, Cas9 and B2M sgRNA for up to 45 minutes. The line graph shows iTOP efficiency over time.

FIG. 4: Effects of glycerol on transduction efficiency in hiPSCs

hiPSCs were incubated with NT2100 or NT2100.2 transduction buffer, sp-cas9-1×NLS and a sgRNA targeting CD55, as described in Example 5. Cells were incubated with transduction mix for 15 minutes. CD55 knockout was assessed via ICE and FACS.

FIG. 5: iTOP-mediated delivery of CRISPR/Cas9 in adherent and suspension cells

Workflow demonstrating the iTOP protocol for transduction into cells and the assessment of post-iTOP cell viability, recovery and editing efficiency.

FIG. 6: iTOP-mediated B2M editing in ARPE-19 and HEK293 cells with control buffer and newly developed iTOP reagents

Histograms showing B2M editing in A) ARPE-19 cells and B) HEK293 cells after transduction using either control buffer (SB) or newly developed iTOP buffer NT2100. **** depicts a significant difference with P<0.0001. The bars represent standard deviation.

FIG. 7: B2M knockout in T-cells is effective with different Cas9 enzymes

CD4+ T cells were incubated with NT2100 buffer and a 1:1 ratio of synthetic B2M ctRNA and spCas9 (4×NLS-SpCas9-2×NLS or SpCas9-1×NLS). The histogram shows percentage of B2M knockout measured 4 days post-transduction using FACs (measuring MHC-1 surface expression).

FIG. 8: Glycerol and NLSs contribute to increased transduction efficiency in ARPE-19 cells

ARPE-19 cells were incubated with NT2100 buffer, 0.5 M B2M ctRNA, 0.5 UM SpCas9 (SpCas9-1×NLS or 4×NLS-SpCas9-1×NLS) and glycerol at a final concentration of 30 mM or 1397 mM. The histogram shows the percentage of B2M knockout measured 5 days post transduction using FACs (assessing MHC-1 surface expression).

FIG. 9: ATG13 siRNA KD in resting and Activated CD4+ T cells.

A) Western blot depicting if different durations of iTOP (15 min, 30 min) in combination with 1 μM siATG13 can knockdown ATG13. Cells left in an unstimulated resting state or activated overnight with 1 ug/ml plate bound CD3 & 1 ug/ml CD28. Ctrl condition denotes cells treated with neither iTOP nor siRNA. B) Change in levels of ATG13 normalised to Actin; resting relative to no iTOP Ctrl resting condition & activated cells relative to no iTOP active condition. C) Cell viability 1 day post-iTOP as determined by Trypan Blue. Error bars represent SEM from 3 readings.

FIG. 10: iTOP-mediated editing and post-iTOP viability of ARPE-19 and HEK293 cells

A) B2M knockout in ARPE-19 cells and HEK293 cells incubated with iTOP reagent NT2100.2. B2M ctRNA and SpCas9, and analysed by FACS (assessing MHC-1 surface expression). B) Cells from (A) were subjected to a FACS-based assay to determine cell viability 1 day post iTOP. The values were then normalized against the viability of non-iTOPed/untreated cells to express the percentage of relative viability. C) FACS plots of control/untreated and iTOPed ARPE-19 cells with wild-type and B2M-edited populations. D) FACS plots of control/untreated and iTOPed HEK293 cells with wild-type and B2M-edited populations. The gated cell populations correspond to B2M-knockout cells (KO) (lacking MHC-1 surface expression). The bars represent standard deviation.

FIG. 11: iTOP-mediated editing of hiPSCs

A) Histogram showing percentage of CD55-edited cells and corresponding knockout scores in hiPSCs after transduction using NT2100.2 iTOP buffer. B) FACS plots of control/untreated and iTOPed cells with wild-type and CD55-edited populations. The gated cell population corresponds to CD55-knockout cells (KO).

FIG. 12: iTOP-mediated editing, and post-iTOP viability and recovery of Jurkat cells

A) Histogram showing percentage of cells that lost MHC-I expression, reflecting the percentage of B2M-edited cells, analyzed using a FACS-based assay; and the percentage of B2M editing and the corresponding knockout score (analyzed via ICE) B) FACS plots of control/untreated and iTOPed cells with wild-type and B2M-edited populations. The gated cell population corresponds to B2M-knockout cells (KO) (lacking MHC-1 surface expression). C) and D) Cell viability and recovery I day and 4 days post-iTOP, respectively. for cells from (A). Non-iTOPed/untreated cells were used as a control. **** depicts a significant difference (P<0.0001), while ns describes a non-significant difference (P=0.1758). The error bars represent standard deviation.

FIG. 13: iTOP-mediated editing, and post-iTOP viability and recovery of activated primary T cells.

A) Histogram showing the percentage of cells that lost MHC-I expression, reflecting the percentage of B2M-edited cells (FACS), and the percentage of B2M editing and the corresponding knockout score assessed via ICE. B) FACS plots of control/untreated and iTOPed cells with wild-type and B2M-edited populations. The gated cell population corresponds to B2M-knockout cells (KO) (lacking MHC-1 surface expression). C) and D) Cell viability and recovery I day and 4 days post-iTOP for cells from (A). Non-iTOPed/untreated cells were used as a control. * depicts a significant difference (P=0.0353), while ns describes a non-significant difference (P=0.8661). The error bars represent standard deviation.

FIG. 14: iTOP-mediated editing, and post-iTOP viability and recovery of resting primary T cells.

A) Histogram showing the percentage of cells that lost MHC-I expression, reflecting the percentage of B2M-edited cells (FACS), and the percentage of B2M editing and the corresponding knockout score assessed via ICE. B) FACS plots of control/untreated and iTOPed cells with wild-type and B2M-edited populations. The gated cell population corresponds to B2M-knockout cells (KO) (lacking MHC-1 surface expression). C) and D) Cell viability and recovery 1 day and 4 days post-iTOP, respectively, for cells from A. Non-iTOPed/untreated cells were used as a control. **** and ** depict a significant difference with P<0.0001 and P=0.0036, respectively. The error bars represent standard deviation.

FIG. 15: Comparison of editing efficiency, viability and recovery of Jurkat cells after iTOP and electroporation.

A) Histogram showing the percentage of cells that lost MHC-1 expression, reflecting the percentage of B2M-edited cells, ns represents a non-significant difference (P=0.7267). B) and C) Directly after iTOP, cells were maintained in medium with post-iTOP viability enhancer. Histograms show cell viability and recovery assessed via FACs. The values were normalized against the viability and recovery of non-iTOPed/untreated cells to express the percentage of relative viability and recovery. **** depicts a significant difference (P<0.0001). The error bars represent standard deviation.

FIG. 16: B2M knockout in ARPE-19 and HEK293 cells after iTOP, and lipofection.

Histogram showing the percentage of cells that lost MHC-I expression, reflecting the percentage of B2M-edited cells. ** and **** represent a significant difference with P=0.0027 and P<0.0001, respectively. The error bars represent standard deviation.

Table 1:

List of transduction compounds their protein transduction activity and effect on cell proliferation in transduction buffer. First column: transduction compound number; Second column: chemical structure of the transduction compound. Third column: Relative β-lactamase protein transduction activity; Fourth column: Relative BrdU incorporation 24 hrs after β-lactamase transduction. MEFs were transduced for 3 hours with 1 μM β-lactamase protein at a NaCl adjusted osmolality of 700 mOsm/Kg in transduction buffer containing 30 mM of Glycerol and 15 mM of Glycine and 25 mM of the indicated transduction compounds. β-lactamase incorporation of the reference compound (NDSB201, #22) was set at 100%. Open circles indicate relative BrdU incorporation by the transduced cells. BrdU incorporation of untransduced cells was set at 100% and BrdU incorporation of mitomycin C-treated cells was set at 0%.

### Example 1: Exemplary Transduction Buffers of the Invention

Transduction buffers NT2100 and NT2100.2 were developed with the aim to improve transduction efficiency in difficult to transduce cell types.

Table A below describes the composition of transduction buffers NT2100, NT2100.2 and SB (control) buffer.

TABLE A

| | NT2100 | NT2100.2 | SB (control) | |
|---|---|---|---|---|
| | buffer | buffer | buffer | |
| GABA | 250 mM | 250 mM | 252 | mM |
| Glycine | 15 mM | 15 mM | 15 | mM |
| Glycerol | 30 mM | 1530 mM | 30 | mM |
| Serine | 150 mM | 150 mM | — | |
| NaCl | 800 mM | 800 mM | 424 | mM |
| Trehalose | 50 mM | 50 mM | — | |
| MgCl₂ | 1.25 mM | 1.25 mM | 0.25 | mM |
| Phosphate buffer, pH 8.0 | 5 mM | 5 mM | 5 | mM |
| Glutamine | — | — | 1.5 | mM |
| NEAA: glycine, L-alanine, | — | — | 75 μM of each | |
| L-asparagine, L-aspartic | | | amino acid | |
| acid, L-glutamic acid, | | | | |
| L-proline, L-serine. | | | | |
| N2: | — | — | 7.5 | μM |
| Human transferrin (Holo) | | | | |
| Insulin recombinant Full Chain | | | 0.65 | μm |
| Progesterone | | | 15 | nm |
| Putrescine | | | 75 | μm |
| Selenite | | | 22.5 | nm |

| | | | |
|---|---|---|---|
| B27 | — | — | 0.75× |
| Optimem | — | — | To dissolve |
| | | | components in |

### Example 2: Transduction Efficiency in Human Induced Pluripotent Stem Cells

For transduction (iTOP), hiPSCs were incubated with iTOP mix comprising SB or NT2100.2 transduction buffer, spCas9-1×NLS and a sgRNA targeting beta 2 microglobulin (B2M), resulting in abrogation of MHC1 surface expression. For hiSPCs, 15 UM Cas9 and sgRNA can be used. Cells were incubated with the iTOP mixture for 15 min (NT2100.2; SB) or 45 min (SB).

### Results

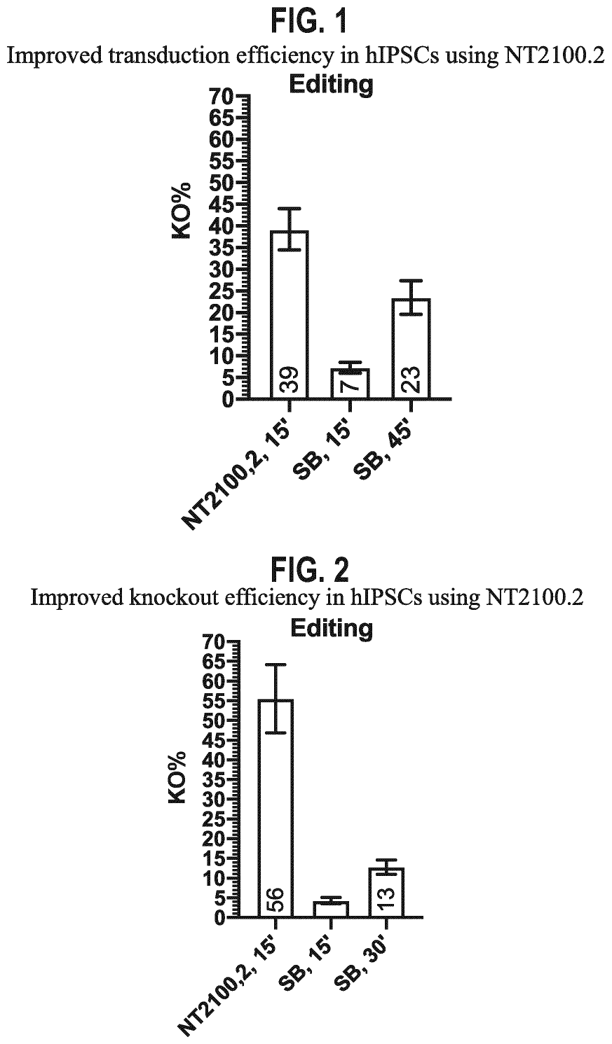

FIG. 1 shows the percentage knockout of B2M in hiPSCs following transduction with transduction compositions SB or NT2100.2.

For cells transduced using SB buffer, 7% knockout of B2M was achieved after 15 minutes incubation, and after 45 minutes 23% knockout was achieved.

Using NT2100.2 buffer, which contains serine, trehalose and glycerol, it took only 15 minutes to achieve knockout of B2M in 39% of cells.

Thus it was concluded that the efficiency of knockout efficiency following transduction can be dramatically improved using transduction buffer NT2100.2

### Example 3: Transduction Efficiency in Human Induced Pluripotent Stem Cells

The assay described in example 2 was repeated, with incubation periods of 15 min (NT2100.2 (n=2) and SB (n=2)) or 30 min (SB).

### Results

The results of this experiment are shown in FIG. 2. 15 min incubation with NT2100.2 resulted in improved editing efficiency (56%) when compared to 15 min incubation with SB (approx. 5%). Increasing the incubation with SB buffer to 30 min did not result in the same amount of editing as 15 min incubation with NT2100.2 (13% SB vs 56% NT2100.2).

The results demonstrate the remarkable efficiency of CRISPR/Cas9 gene editing using NT2100.2.

### Example 4: Transduction Efficiency in Primary T Cells

Primary T cells were incubated with iTOP mix comprising NT2100, NT2100.2 or SB buffer, 20 UM Cas9 and 20 μM B2M sgRNA for up to 45 minutes.

### Results

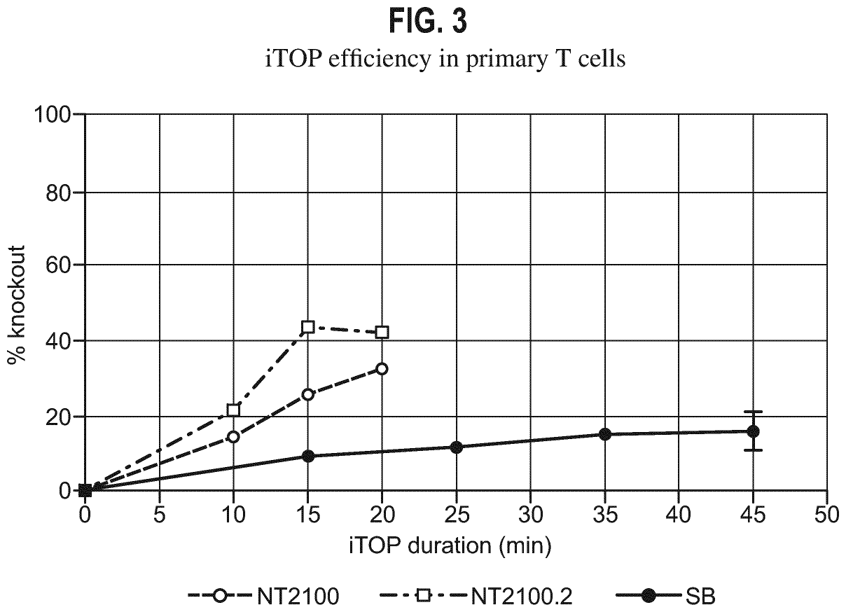

The results of this experiment are shown in FIG. 3. After 15 min incubation with SB <10% of cells achieved B2M knockout, as measured by FACS analysis of MHC1 surface expression. After 45 min incubation with SB, B2M knockout was achieved in less than 20% of cells. In contrast, 15 min incubation using NT2100 transduction buffer achieved B2M knockout in 25% of cells. Use of NT2100.2 led to an even greater knockout efficiency, with over 40% knockout achieved in 15 min.

Thus, these results indicate that buffers NT2100 and NT2100.2 can improve knockout efficiency in primary T cells.

### Example 5: Addition of Glycerol can Further Improve Transduction Efficiency in hIPS Cells

For transduction, hiPSCs were contacted with iTOP mix comprising NT2100 or NT2100.2, sp-Cas9-1×NLS, and a sgRNA targeting CD55. NT2100.2 contains all the components of NT2100, and an additional 1500 mM glycerol. Cells were incubated with the transduction mixture for 15 min.

Following the incubation period, knockout efficiency was assessed using flow cytometry (FACs) and Inference of CRISPR Edits (ICE).

### Results

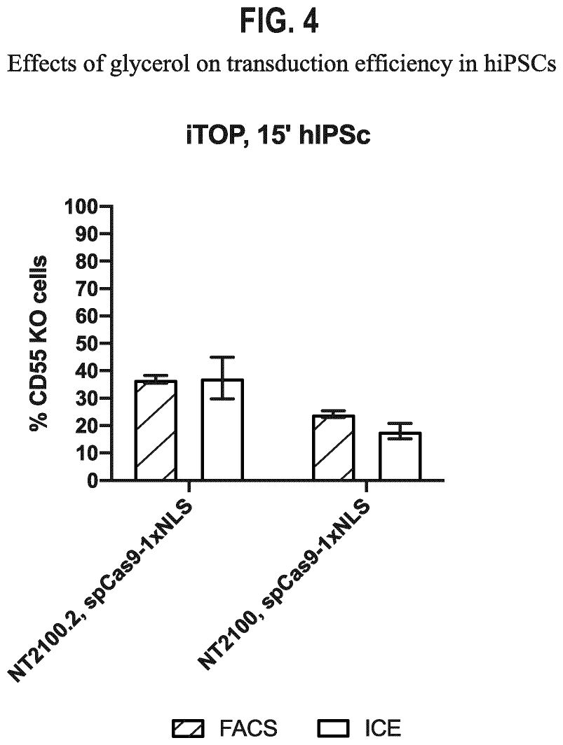

The results of this experiment are shown in FIG. 4. 15 min incubation with NT2100.2 led to improved gene knockout (about 40% measured by FACs and ICE) when compared to use of NT2100 (about 25% as assessed by FACs; about 20% as assessed by ICE).

This demonstrates that the addition of glycerol can improve transduction efficiency in hiPSCs.

### Example 6. Optimized Protocol for iTOP-Mediated Delivery of CRISPR/Cas9 in Adherent and Suspension Cells

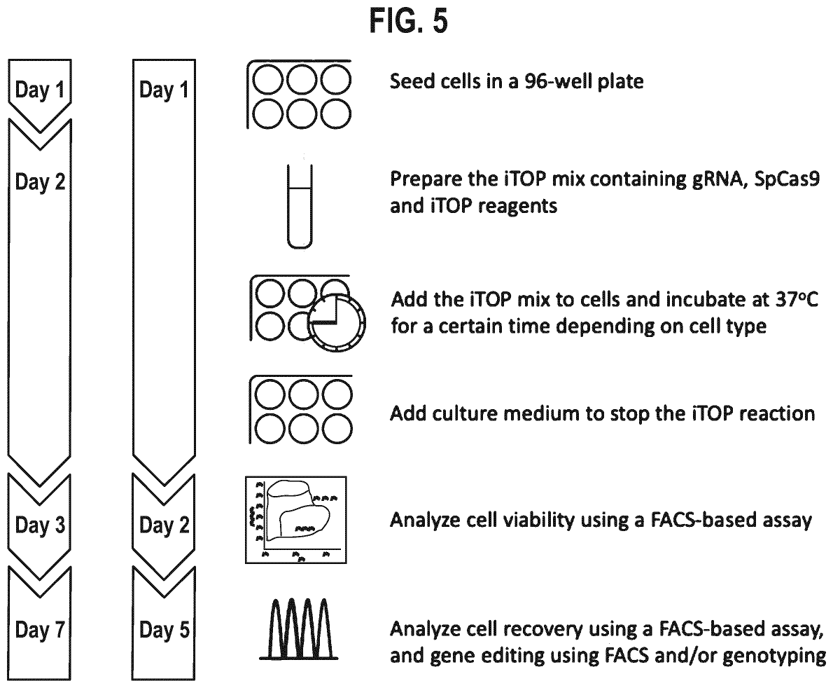

The inventors have optimized the protocol for iTOP-mediated delivery of CRISPR/Cas9 to ensure a simple, fast and efficient method for the introduction of CRISPR/Cas9 into a variety of adherent and suspension human cells. The general protocol is shown in FIG. 5.

The composition of iTOP reagents NT2100 and NT2100.2 (see Table A) was developed and compared to the previously developed buffer SB.

ARPE-19 cells were incubated for 45 min or 120 min with iTOP mix containing 0.5 μM B2M ctRNA, 0.5 μM SpCas9 and either the SB buffer or the newly optimized NT2100 buffer. 5 days post-iTOP, cells were analyzed using a FACS-based assay to determine the percentage of cells that lost MHC-I expression, reflecting the percentage of B2M-edited cells. Further details of the methods used are provided in Example 15.

HEK293 cells were incubated for 45 min with iTOP mix containing 15 μM in-vitro transcribed B2M gRNA, 15 μM SpCas9 and either the SB buffer or the newly developed NT2100 buffer. 5 days post-iTOP, cells were lysed for genomic DNA isolation and subsequent sequencing and ICE analysis to determine the percentage of B2M editing and the corresponding knockout score. Further details of the methods used are provided in Example 15.

### Results

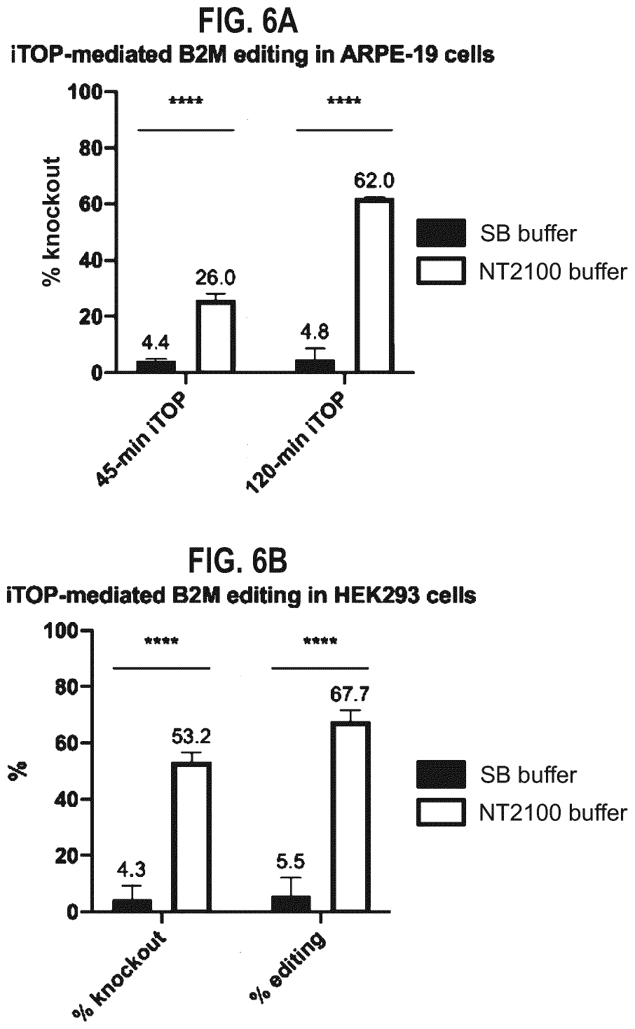

The results of this experiment are shown in FIG. 6. In ARPE-19 cells, 45 min incubation with NT2100 resulted in improved B2M knockout (26%) when compared to 45 min incubation with SB (4.4%) (FIG. 6A). Increasing the incubation time to 120 minutes resulted in 62% B2M knockout using NT2100 buffer (FIG. 6A). In contrast, only 4.8% B2M knockout was achieved following 120 min incubation with SB buffer (FIG. 6A).

In HEK293 cells, 45 min incubation with NT2100 resulted in 53.2% B2M knockout, compared to only 4.3% knockout using SB buffer (FIG. 6B). After 120 min incubation, 67.7% knockout was achieved using NT2100 buffer, while incubation with SB buffer resulted in only 5.5% knockout (FIG. 6B).

Thus, these results indicate that NT2100 buffer can significantly improve knockout efficiency in ARPE-19 and HEK293 cells.

### Example 7: Transduction of Different Types of Modified Cas9 Nucleases

Freshly isolated CD3/CD28-activated CD4 T cells were incubated with iTOP mix comprising 1:1 ratio B2M ctRNA and SpCas9 (SpCas9-1×NLS or 4×NLS-SpCas9-1×NLS) and NT2100 buffer. Cells were maintained in RPMI-based medium (No. 61/870,044) containing 5% human serum (HS), 1% penicillin-streptomycin (P/S), 1% sodium pyruvate and 1% NEAA. The FACS-based measurement of MHC-1 surface expression (indicating B2M editing) was done 4 days post-iTOP.

### Results

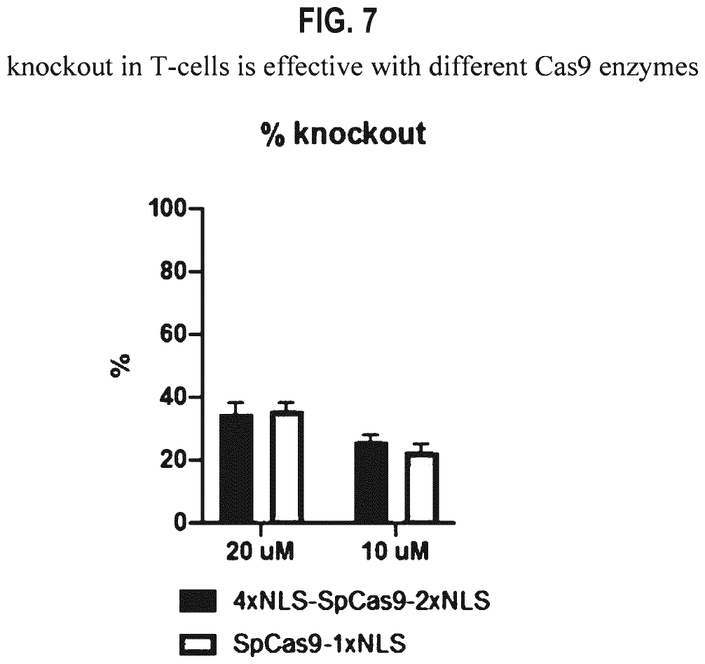

The results of these experiments are shown in FIG. 7. The data shows that efficient transduction was achieved for both SpCas9-1×NLS and 4×NLS-SpCas9-1×NLS. At high concentrations of ctRNA and SpCas9 (20 μM), transduction efficiency for SpCas9-1×NLS and 4×NLS-SpCas9-1×NLS was the same. However, at lower concentrations (10 μM) transduction efficiency was slightly higher when 4×NLS-SpCas9-1×NLS was used.

### Example 8: Effect of NLSses and Glycerol in ARPE-19 Cells

ARPE19 cells were incubated for 60 min with iTOP mix comprising 0.5 μM B2M ctRNA, 0.5 UM SpCas9 (SpCas9-1×NLS or 4×NLS-SpCas9-1×NLS) and NT2100 supplement, with glycerol added to a final concentration of 30 mM or 1397 mM. Cells were cultured with DMEM/F12 based medium (#31331028) containing 10% fetal bovine serum (FBS) and 1% penicillin-streptomycin (P/S). The FACs-based measurement of MHC-1 surface expression (reflecting B2M knockout) was determined 5 days post-iTOP.

### Results

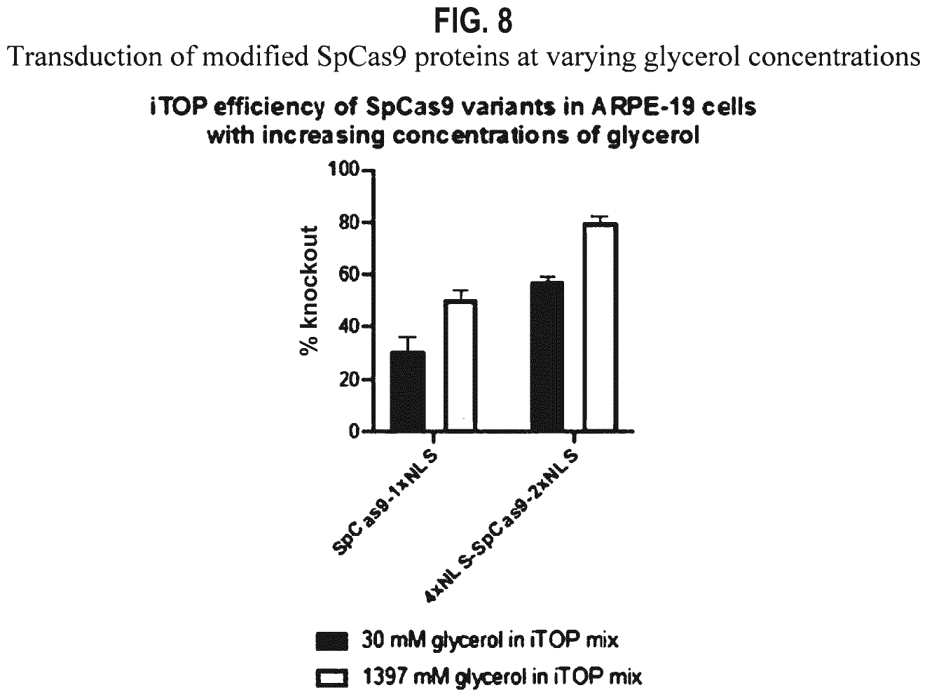

The results of these experiments are shown in FIG. 8. Use of 4×NLS-SpCas9-1×NLS increased B2M knockout efficiency in ARPE-19 cells. When using NT2100 buffer and low levels of glycerol, almost 60% B2M KO was achieved using 4×NLS-SpCas9-1×NLS, compared to 30% B2M KO achieved using SpCas9-1×NLS. A low concentration of SpCas9 was used in this experiment (0.5 μM), confirming the findings of Example 7 that 4×NLS-SpCas9-1×NLS offers improved transduction efficiency when using low concentrations of SpCas9.

The addition of relatively high levels of glycerol to the transduction mix also further improved transduction efficiency in ARPE-19 cells. When SpCas9-1×NLS was used, additional glycerol increased B2M KO from 30% to 50% (FIG. 8). Similarly, when using 4×NLS-SpCas9-1×NLS additional glycerol improved B2M KO from 60% to 80% (FIG. 8).

### Example 9: ATG13 siRNA KD in Resting and Activated CD4+ T Cells

The aim of this experiment was to test whether iTOP transfection reagent can be used for siRNA knockdown in resting human CD4+ T cells. This example used siRNA alone, to test whether the transduction buffers of the invention can be useful for RNA interference, as well as gene editing.

Isolation and pre-iTOP Culture

99 mL blood obtained from UMCU MDD was used for isolation of CD4+ T cells. Cells were diluted 1:1 in PBS and loaded onto 15 ml ficol for PBMC separation. Ficol separation centrifugation was performed at 2200 rpm for 20 min with no brake. Following centrifugation, the PBMC layer was harvested and washed 2× in 50 mL cold MACs Buffer (2% FBS and 4 mM EDTA). Negative isolation was performed using a MagniSort™ Human CD4 T cell enrichment kit (Invitrogen 8804-6811-74), according to the manufacturers protocol.

˜45 million CD4+ cells were obtained from ˜200 million PBMCs. Half of the cells were left in a resting state overnight at a density of 1 million cells p/mL, in cell medium (RPMI 1640 and Glutamax™, 10% FBS, 1% P/S). The remaining half of the cells were activated with plate bound CD3 (1 μg/mL) and CD28 (1 μg/mL) overnight at a density of 1 million cells p/mL.

iTOP Reaction:

42 million isolated CD4+ cells obtained as above were split between 6 conditions (7 million cells per condition):
No iTOP Ctrl, No siRNA, Resting
No iTOP Ctrl, No siRNA, Activated
15 min iTOP, 1 uM siATG13, Resting
15 min iTOP, 1 uM siATG13, Activated
30 min iTOP, 1 uM siATG13, Resting
30 min iTOP, 1 uM siATG13, Activated

Cells were transferred and pelleted in a 15 mL tube (centrifugation 5 min, 500×g). Medium was removed, and cells were centrifuged again (1 min, 500×g), and all remaining medium was removed manually via pipette.

For each iTOP reaction cells were resuspended in 300 μL iTOP mix containing 35 μL H₂O, 20 μL NTrans protein buffer, 40 μL NT2100 supplement, to make a NT2100 buffer as per Table A above, and 5 μL siATG13 (from 20 μM stock solution). Reactions were carried out according to their experimental time course, and were stopped by the addition of 5 mL cell medium (as described above).

Following iTOP reactions, cells were pelleted via centrifugation (5 min, 500×g), the supernatant was removed and cells were plated at a density of ˜1 million cells p/mL medium.

Cell viability was assessed one day post-iTOP using Trypan Blue. Cells were harvested ˜60 hr post iTOP to assess knockdown efficiency using Western blot.

### Cell Lysis and Protein Quantification

Cells were lysed in 20 μL RIPA buffer (150 mM NaCl, 1% Triton, 0.5% Sodium Deoxycholate, 0.1% SDS, 50 mM Tris, pH 8.0 (+1× Halt Protease Inhibitor cocktail fresh+1× Orthovanadate phosphatase inhibitor fresh)) for 30 min on ice.

Cell membranes were pelleted by centrifugation at 13200 rpm for 10 min, supernatant was transferred to a new Eppendorf and stored at −20° C. until use.

### Bradford Protein Quantification

A BSA concentration curve was used to quantify the protein concentrations. The BSA concentration curve was created according to Table 2 below. Reagents used for creating this curve were 0.137 μg/μL BSA Solution, 1.37 μg/μL BSA Solution and Bio-rad Protein Assay Dye Reagent Concentrate (#5000006).

TABLE 2
BSA concentration curve for assessing protein
concentration in samples following iTOP
BSA Concentration Curve:

| Concen- | | | | | | | | |
|---|---|---|---|---|---|---|---|---|
| tration | | | | | | | | |
| ug/ml: | 0.5 | 1 | 2.5 | 5 | 10 | 20 | 50 | 100 |
| ul from | (0.137 ug/ul) | | | | (1.37 ug/ul) | | | |
| BSA Stock | 3.65 | 7.3 | 18.25 | 3.65 | 7.3 | 14.6 | 36.5 | 73 |
| ul of H₂O | 96.35 | 92.7 | 81.75 | 96.35 | 92.7 | 85.4 | 63.5 | 27 |

700 μL of H₂O was added to each tube for a total of 800 μL. 2 μL of each sample to be tested was quantified in 800 μL H₂O. 200 μL Protein Assay Dye Reagent Concentrate was added to each sample, the samples were vortexed and incubated for 10 min. Samples were then transfer to 1 mL cuvettes to measure absorbance at 600 nm using UV visible spectroscopy.

### Western Blotting

5× sample buffer was made up consisting of: 10% SDS, 50% Glycerol, 10% β-mercaptoethanol, 300 mM Tris (pH 6.8) and ˜0.1% Bromophenol Blue.

Samples were run on a 1.5 mm 10% acrylamide gel, with 20 μg sample loaded per well. Transfer was performed on turboblot, using 30 min protocol (25 V, 1 A). Following transfer, the membrane was cut at the 55 kDa ladder, and the top half stained for ATG13 and lower half stained for Actin, using the antibodies as described in Table 3 below.

TABLE 3
Antibodies used for Western Blot

| Antibody: | Dilution: | Secondary: | Dilution |
|---|---|---|---|
| Atg13 (D4P1K), | 1:1000, 3% | Polyclonal swine | 1:5000, 5% |
| Rabbit, Cell | BSA/TBST | anti-rabbit, | ELK/TBST |
| Signalling | | immunoglobulin-HRP, | |
| Technology ®, | | Dako ® P0217 | |
| 13273 | | | |
| Actin, Goat | 1:1000, 3% | Polyclonal rabbit | 1:5000, 5% |
| Polyclonal, | BSA/TBST | anti-goat, | ELK/TBST |
| Santa Cruz ® | | immunoglobulin-HRP, | |
| sc-1616 | | Dako ® P0160 | |

### Results

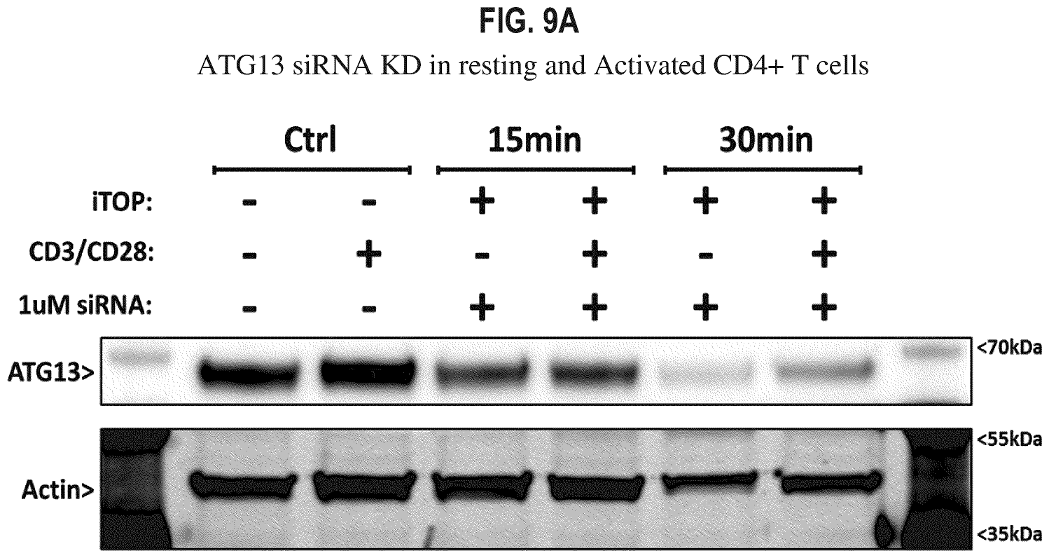

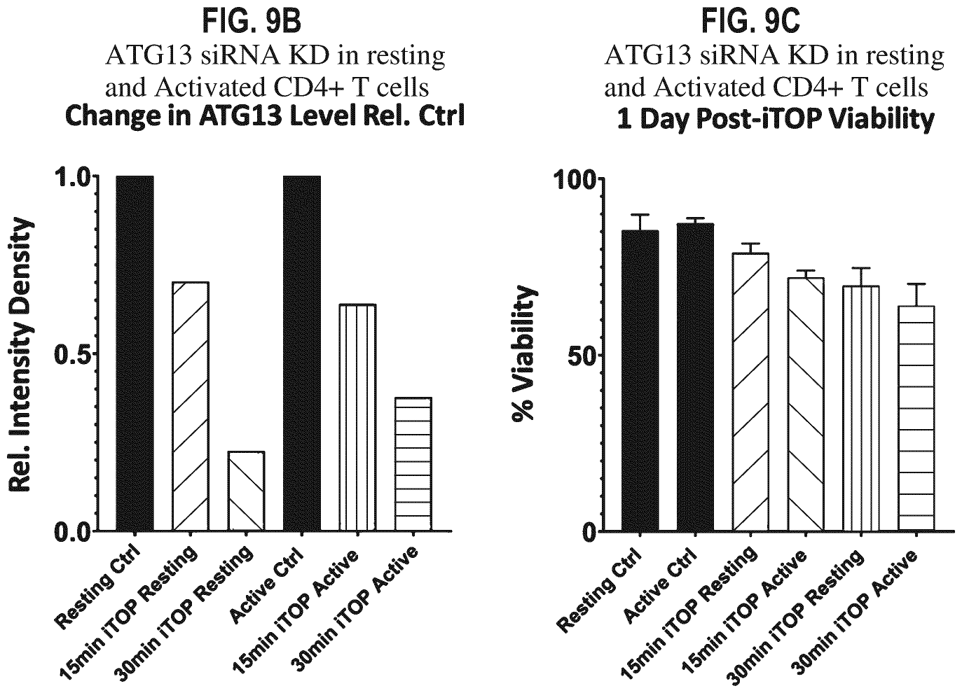

FIG. 9 shows the change in ATG13 level relative to control in resting and activated CD4+ cells following incubation with NT2100 buffer and siRNA targeting ATG13. Levels of actin in the samples were used as a baseline for normalisation.

For resting CD4+ cells, 15 min incubation in the iTOP mix resulted in a 30% reduction in ATG13 protein levels (FIG. 9B). Increasing the incubation time to 30 min resulted in a remarkable reduction of in ATG13 levels, of around 80%. Knockdown of ATG13 was also achieved in active CD4+ cells, with a 35% reduction in ATG13 compared to the control after 15 min iTOP, and a 65% reduction after 30 min iTOP.

1-day post iTOP, the viability of CD4+ cells was slightly reduced compared to untreated control cells. However, viability remained above 60% for all treatments.

These results demonstrate that NT2100 buffer allows efficient transduction of siRNA in CD4+ cells, allowing significant knockdown of a target.

### Example 10. High iTOP-Mediated Editing Efficiency with CRISPR/Cas9 in Adherent Cell Lines

For evaluating the iTOP-mediated editing efficiency in adherent cells, iTOP reactions were performed to target B2M with CRISPR/Cas9 in two common adherent cell lines: ARPE-19 and HEK293. ARPE-19 and HEK293 cells were incubated for 60 min or 45 min, respectively, with NT2100.2 (APRE-19 cells) or NT2100 (HEK293 cells) containing a concentration range of B2M ctRNA and SpCas9 in 1:1 molar ratio. 1 day after iTOP, cell viability was measured using flow cytometry, while 5 days after iTOP both cell viability (i.e. recovery) and loss of MHC-I expression were determined with flow cytometry (reflecting B2M editing). Further details of the methods used are provided in Example 15.

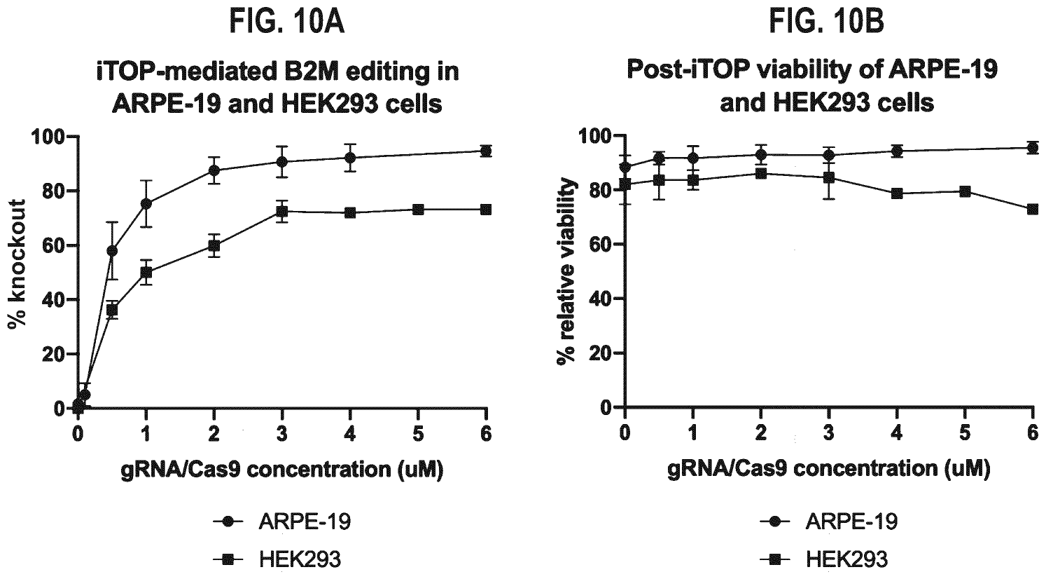

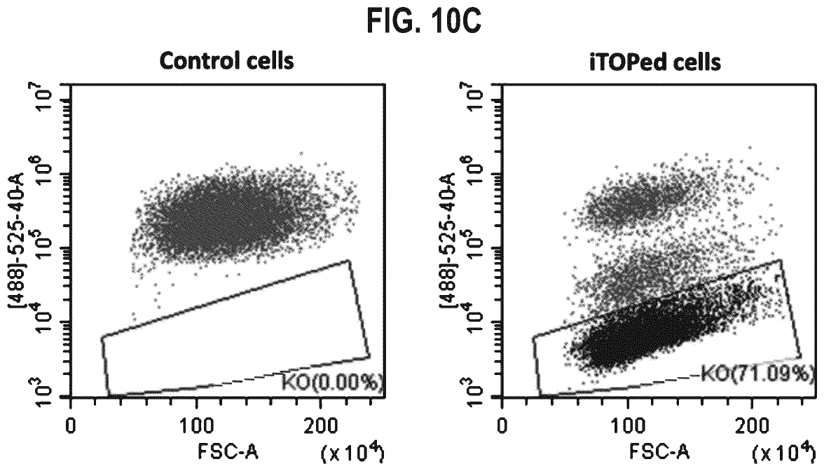

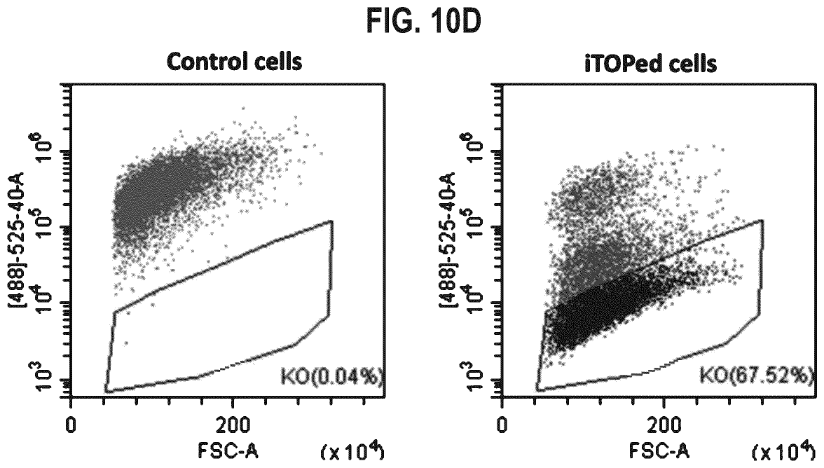

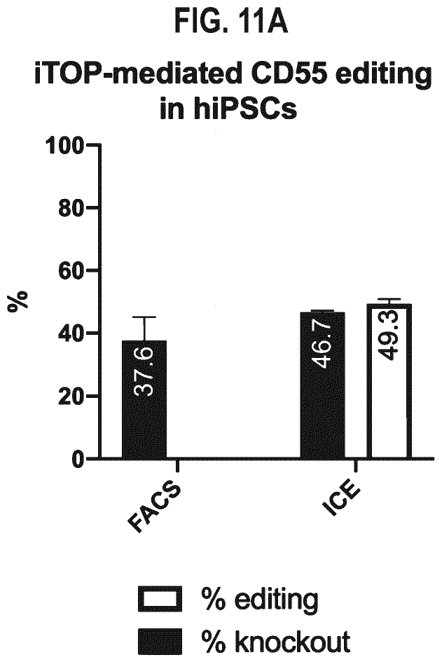

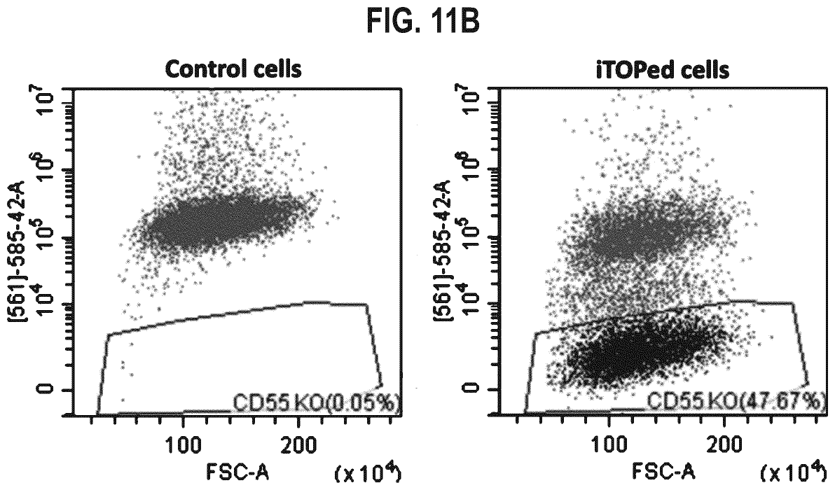

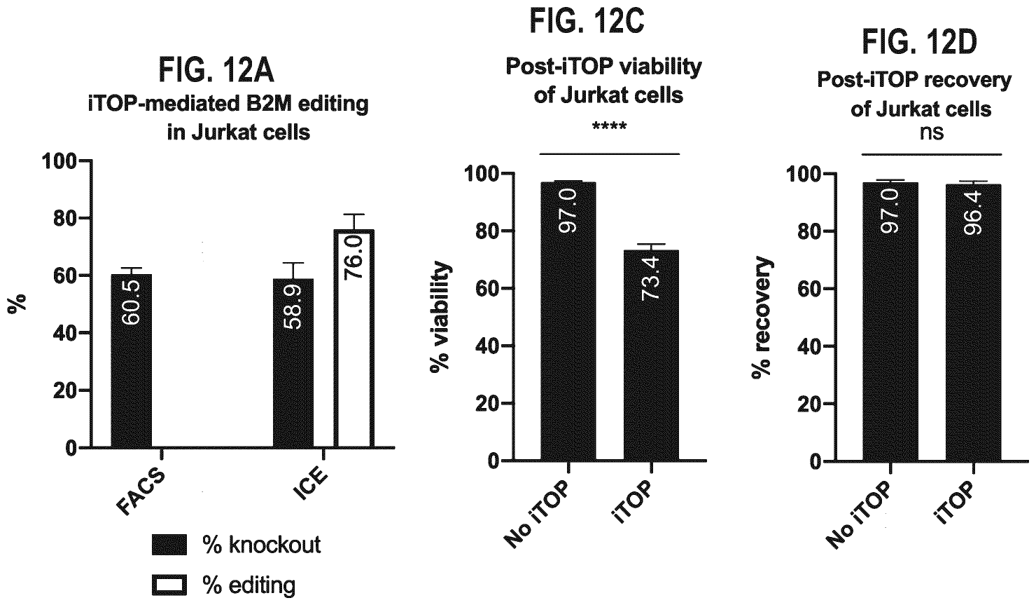

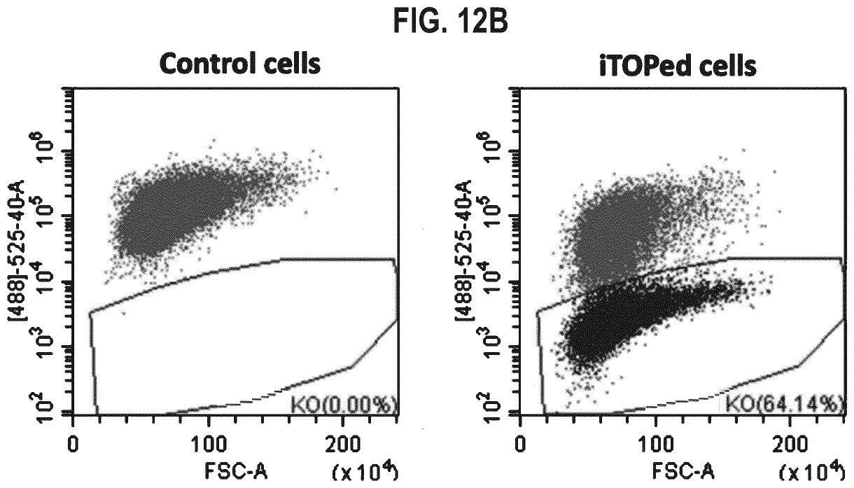

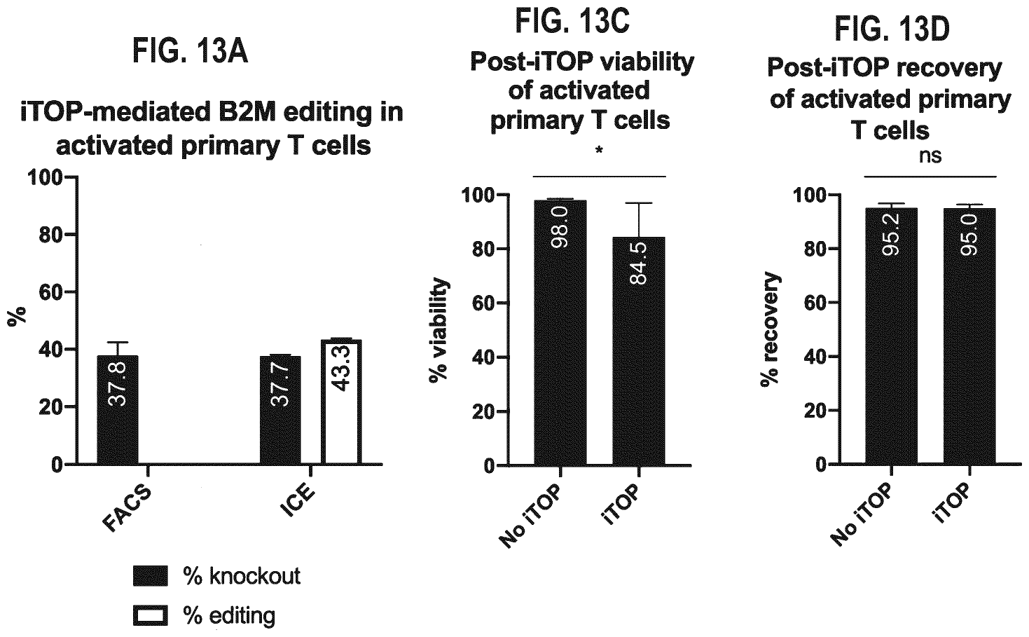

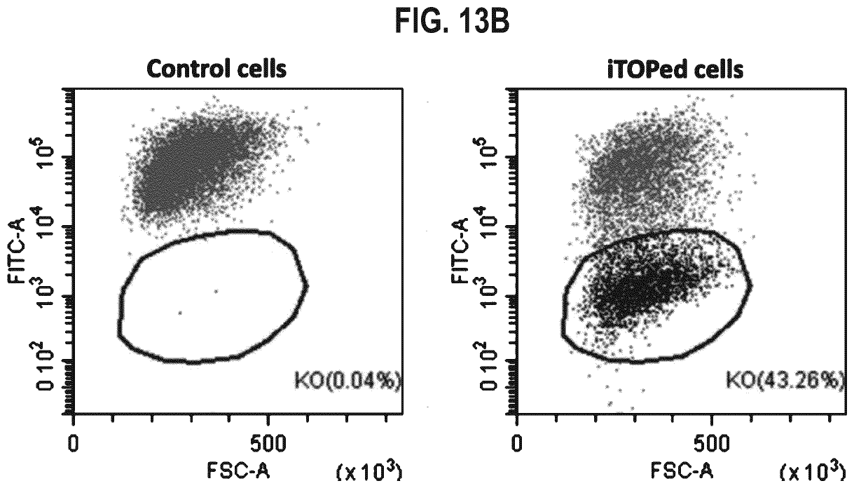

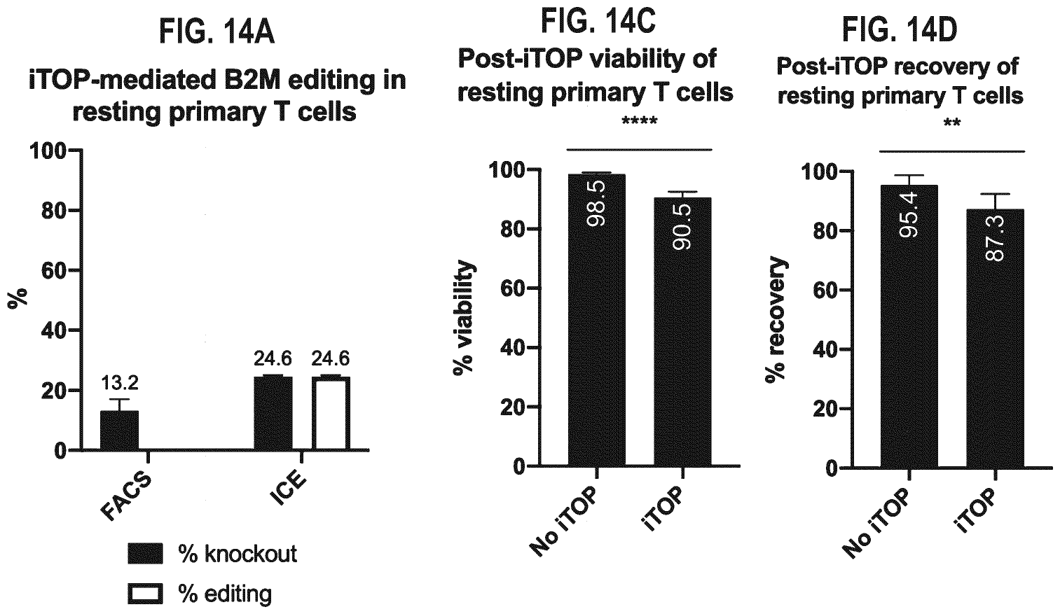

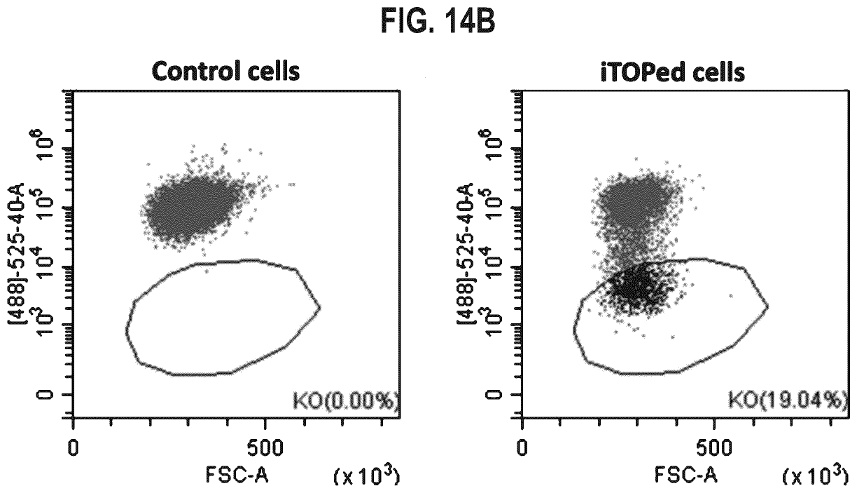

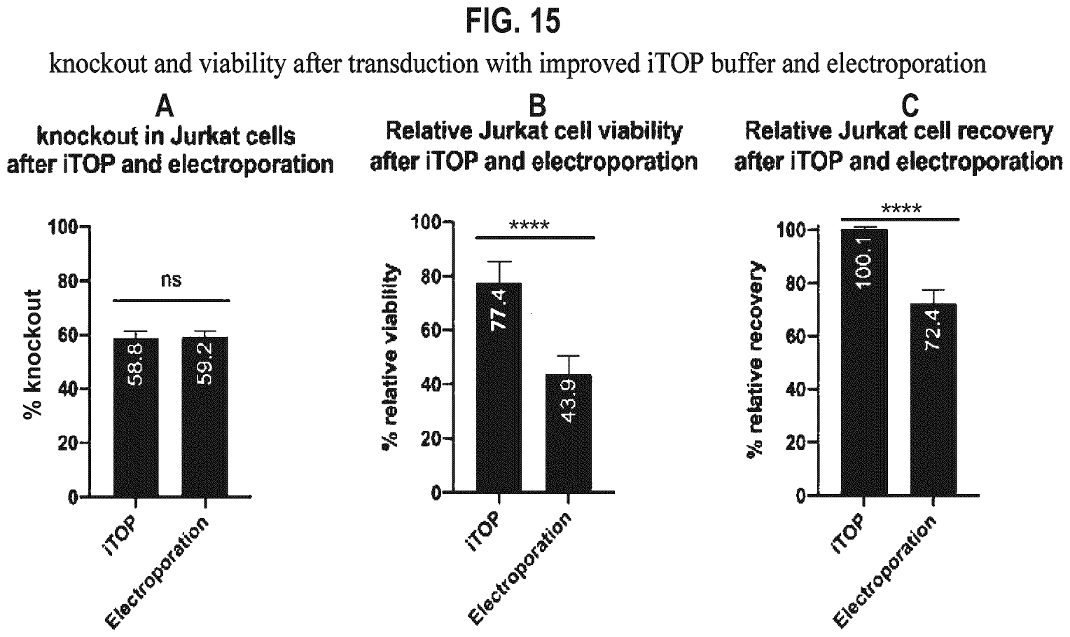

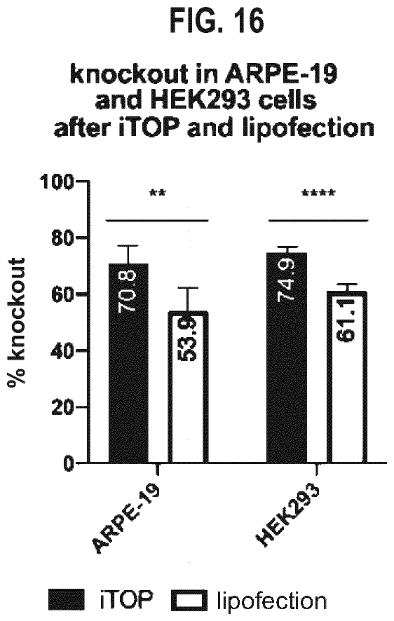

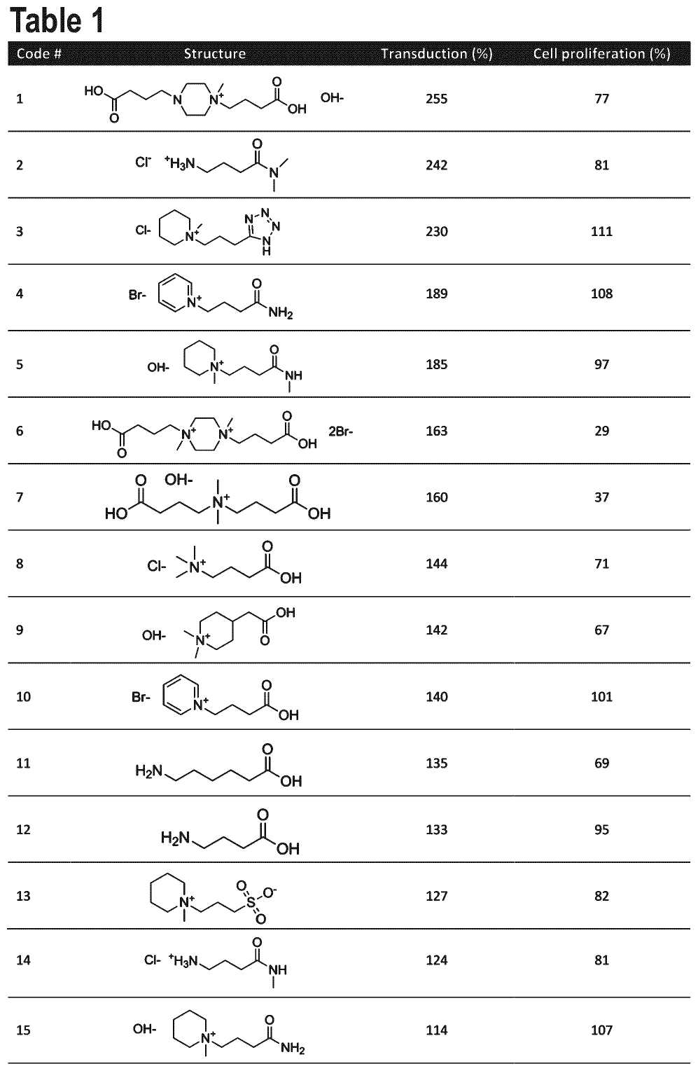

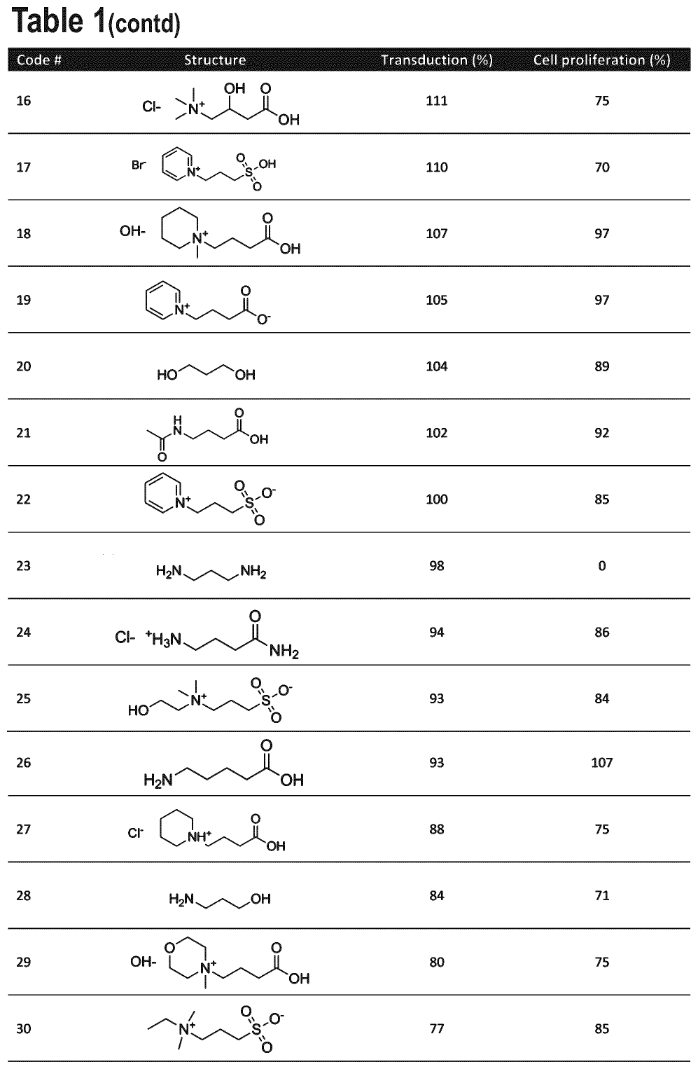

Results showed that the iTOP-mediated editing efficiency, expressed as loss of MHC-I from the cell surface, improved with increasing concentrations of B2M ctRNA and SpCas9 (FIG. 10A). The plateau in editing efficiency and B2M knockout was already achieved with 3 μM B2M ctRNA/SpCas9, establishing this concentration as optimal in ARPE-19 and HEK293 cells. With this concentration, ˜85% B2M knockout in ARPE-19 cells was observed, while ˜70% B2M knockout was achieved in HEK293 cells (FIG. 10A). Interestingly, a certain percentage of both ARPE-19 and HEK293 cells had also partially lost their MHC-I expression, most likely due to the editing of only one B2M allele. Therefore, they appeared in FACS plots as -knockdown cells between the wild-type and knockout cells (FIGS. 10C and D). Post-iTOP viability of ARPE-19 and HEK293 cells remained high at ˜80-90% (FIG. 10B). However, with increasing concentrations of B2M ctRNA and SpCas9, HEK293 cell viability slightly decreased, while ARPE-19 cell viability remained unaffected, most likely due to ARPE-19 cells being more robust and resistant to various treatments than HEK293 cells (FIG. 10B).

### Example 11. Successful iTOP-Mediated Editing of Adherent hiPSCs

To assess the efficiency of iTOP-mediated delivery of CRISPR/Cas9 in difficult-to-transduce hiPSCs, iTOP reactions targeting the CD55 gene (Wu et al., Stem Cell Res, 29, 2018, 6-14) were performed. CD55 targeting was chosen, because hiPSCs have well detectable CD55 expression, while their MHC-I expression is too low for reliable quantification with flow cytometry. hiPSCs were incubated for 15 min with iTOP reagent NT2100.2 containing optimal concentrations of CD55 ctRNA and SpCas9 as determined in these cells (i.e. 15 μM, data not shown). 5 days after iTOP, loss of CD55 expression was measured with flow cytometry, while CD55 editing was also assessed with sequencing followed by ICE analysis. Further details of the methods used are provided in Example 15.

Results showed that ˜40% of cells did not express CD55 and that CD55 was edited up to ˜45-50% according to ICE analysis, revealing that flow cytometry and ICE analysis provide similar results (FIG. 11A). Interestingly, a small number of cells appeared in FACS plots between the wild-type and CD55-knockout populations, most likely because only one allele of CD55 was edited in those cells (FIG. 11B).

### Example 12. Efficient iTOP-Mediated Delivery of CRISPR/Cas9 in Suspension Jurkat and Primary Human T Cells

To determine the efficiency of iTOP-mediated delivery of CRISPR/Cas9 in suspension human T cells, iTOP reactions were performed to target B2M in Jurkat cells and the difficult-to-transduce primary CD4+ T cells. Jurkat and CD4+ T cells were incubated for 45 min or 15 min, respectively, with the iTOP reagent NT2100.2 containing optimal concentrations of B2M ctRNA and SpCas9 as determined in these cells (i.e. 20 μM, optimization data not shown). 1 day after iTOP, cell viability was measured using flow cytometry. 4 days after iTOP both cell viability (i.e. recovery) and loss of MHC-I expression were determined with flow cytometry (reflecting B2M editing), while B2M editing was also assessed with sequencing and subsequent ICE analysis. Further details of the methods used are provided in Example 15.

In Jurkat cells, it was observed that ˜60% of cells had lost MHC-I expression (reflecting B2M knockout), as determined by flow cytometry (FIGS. 12A and B). Sequencing and ICE analysis confirmed the knockout score, revealing that results from flow cytometry and ICE analysis are comparable. ICE analysis additionally showed, however, that B2M editing was higher than the B2M knockout levels (FIG. 12A). Most likely, this is due to the fact that not all editing events in B2M gene led to frameshift mutations resulting in loss of MHC-I expression. Apart from high editing. Jurkat cells also had high viability and recovery percentage. 1 day after iTOP, they showed ˜70% viability (FIG. 12C), while 4 days after iTOP they fully recovered and had the same viability as the untreated control cells (FIG. 12D), indicating that Jurkat cells can also undergo iTOP reactions successfully. When examining the iTOP-mediated B2M editing in anti-CD3/CD28-activated CD4+ T cells with flow cytometry, we found that ˜40% of cells did not express MHC-I, indicating ˜40% B2M editing (FIGS. 13A and B). On a genomic level, sequencing and ICE analysis confirmed these results (FIG. 13A), demonstrating once more that results from flow cytometry and ICE analysis are similar. As determined by flow cytometry, activated CD4+ T cells remained viable at a high percentage 1 day after iTOP (˜80% viability) (FIG. 13C), and they fully recovered 4 days after iTOP (˜95% viability) (FIG. 13D). Their viability 4 days after iTOP was the same as of the untreated control cells, suggesting that cell growth and expansion were not affected negatively by iTOP reactions. To determine whether T-cell activation affects the iTOP editing efficiency and post-iTOP cell viability, the same iTOP reaction was performed in resting CD4+ T cells as was previously performed in activated CD4+ T cells. It was found that ˜20% of resting cells did not express MHC-I (reflecting B2M knockout) (FIG. 14A). Interestingly, a small percentage of cells had partially lost MHC-I expression as well and appeared in FACS plots between the wild-type and the edited cell populations (FIG. 14B). Most likely, this occurred due to the editing of one of the alleles of the B2M gene. Regarding the post-iTOP viability of resting cells, flow cytometry results showed that I day after iTOP they were ˜90% viable (FIG. 14C). 4 days after iTOP resting cells were still highly viable at ˜85%, however, they were not as viable as the untreated control cells (FIG. 14D). The decreased cell viability over time was expected, since resting CD4+ T cells do not proliferate without prior activation.

### Example 13: Comparison of iTOP-Mediated Delivery to Electroporation

Transduction efficiency and cell viability and recovery were determined for iTOP mediated delivery and the commonly used electroporation method. Difficult-to-transduce Jurkat suspension cells were used for this experiment. Further details of the methods used are provided in Example 15.

iTOP Compared to Electroporation in Suspension Cells

Jurkat cells were incubated for 45 min with iTOP mix comprising NT2100.2 buffer, 15 μM B2M ctRNA and 15 μM SpCas9, or were electroporated using 5 μM B2M ctRNA and 5 μM SpCas9. After iTOP, culture medium with post-iTOP viability enhancer (Human Serum at 5% (Sigma, H3667, heat inactivated)) was added to iTOPed cells.

Using flow cytometry, the viability of cells was determined 1 day and 4 days after treatment, while the loss of MHC-1 expression (indicating B2M knockout) was measured 5 days after treatment.

### Results

In Jurkat cells B2M editing efficiency was similar for iTOP and electroporation, with ˜60% of cells losing MHC-1 expression (indicating B2M knockout) after transduction (FIG. 15A).

However, the relative viability of cells after iTOP was almost twice as much as the relative viability of cells after electroporation (FIG. 15B). iTOPed cells were ˜80% viable, also due to the presence of post-iTOP viability enhancer in post-iTOP medium, while electroporated cells were only ˜40% viable (FIG. 8B). In the days following iTOP and electroporation, cells continued to recover. In fact, 4 days post-treatment, iTOPed cells were fully recovered with ˜100% viability, while electroporated cells were only ˜70% viable (FIG. 15C).

### Example 14: iTOP-Mediated Delivery in Adherent Cells and Comparison to Lipofection

In this experiment adherent ARPE-19 and HEK293 cells were used. ARPE-19 cells were incubated for 60 min with iTOP reagent NT2100.2 containing 1 μM B2M ctRNA and 1 μM SpCas9. Similarly, HEK293 cells were incubated for 45 min with iTOP reagent NT2100 containing 3 μM B2M ctRNA and 3 μM SpCas9. Using flow cytometry, the editing efficiency of each method, defined as loss of MHC-I expression (indicating B2M editing/knockout), was measured 5 days after treatment. Flow cytometry was also used to determine the viability of cells 1 day after treatment.

Results showed that the editing efficiency was high in both types of adherent cells. In ARPE-19 cells the iTOP-mediated B2M editing was ˜80% while in HEK293 cells the iTOP-mediated B2M editing was ˜65%. When examining the cell viability after iTOP ˜80% of ARPE-19 and HEK293 cells were viable 1 day after iTOP

Since lipofection is one of the most common methods to deliver CRISPR/Cas9 in adherent cells, it was compared to the iTOP-mediated delivery in ARPE-19 and HEK293 cells.

In all reactions a 1:1 molar ratio of B2M ctRNA and SpCas9 was used. In iTOP reactions, however, 1 μM or 3 μM B2M ctRNA/SpCas9 was used, whereas in lipofection 0.15 μM B2M ctRNA/SpCas9 was used, according to manufacturer's instructions. Using the same concentrations of B2M ctRNA/Cas9 in lipofection as in iTOP (i.e. 1 μM or 3 μM) led to lower editing efficiency (data not shown). ARPE-19 cells were incubated for 60 min with iTOP reagent NT2100.2 containing 1 μM B2M ctRNA and 1 μM SpCas9, or were transfected with 0.15 μM B2M ctRNA and 0.15 μM SpCas9 using a lipofectamine-based reagent according to manufacturer's instructions. Similarly, HEK293 cells were incubated for 45 min with iTOP reagent NT2100 containing 3 μM B2M ctRNA and 3 μM SpCas9, or were transfected with 0.15 μM B2M ctRNA and 0.15 μM SpCas9 using the same lipofectamine-based reagent. With flow cytometry, the editing efficiency of each method, defined as loss of MHC-I expression, was determined 5 days after treatment, while the viability of cells was measured 1 day after treatment.

It was found that the editing efficiency in both adherent cell lines—ARPE-19 and HEK293—was higher upon iTOP (˜70-75% B2M-knockout cells) than upon lipofection (˜55-60% B2M-knockout cells) (FIG. 16). Both cell lines were highly viable after lipofection (˜100% viability) and after iTOP (˜85-95% viability).

### Example 15: Further Details of Methods

### Cells

Jurkat (clone E6-1, TIB-152), ARPE-19 (CRL-2302) and HEK293 (CRL-1573) human cell lines were purchased from the American Type Culture Collection (ATCC). The human episomal iPSC line (A18945) was purchased from Thermo Fisher Scientific. Human CD4+ T cells were isolated from peripheral blood mononuclear cells (PBMCs) using the MagniSort™ Human CD4 T cell Enrichment kit (Invitrogen, 8804-6811-74) according to the manufacturer's instructions, and PBMCs were isolated from whole blood of healthy donors using Ficoll® Paque Plus kit (Sigma, GE17-1440-02) according to the manufacturer's instructions.

### Cell Culture

Jurkat cells were maintained in RPMI 1640 medium (Gibco, 61870044) supplemented with 10% fetal bovine serum (FBS) (Sigma, F7524), 1× penicillin-streptomycin (P/S) (Gibco, 15140122), 1 mM sodium pyruvate (Gibco, 11360039) and 1× non-essential amino acids solution (NEAA) (Gibco, 11140035). ARPE-19 and HEK293 cells were cultured respectively in DMEM/F-12 (Gibco, 31331028) and DMEM (Gibco, 31966021) media each supplemented with 10% FBS (Sigma, F7524) and 1× P/S (Gibco, 15140122). hiPSCs were propagated in Essential 8™ medium (Gibco, A1517001) on tissue-culture dishes (FALCON, 353072) coated with Geltrex (Gibco, A1413301) according to the manufacturer's instructions. CD4+ T cells were maintained in RPMI 1640 medium (Gibco, 61870044) supplemented with 5% human scrum (HS) (Sigma, H3667), 1× P/S (Gibco, 15140122), 1 mM sodium pyruvate (Gibco, 11360039) and 1×NEAA (Gibco, 11140035). After thawing, adherent cells were passaged for 2 weeks, while suspension cells were passaged for at least 1 week before using them for experiments. CD4+ T cells were either freshly isolated from whole blood or were thawed at least 1 week before experiments. After isolation, CD4+ T cells were maintained resting or were activated using plate-bound anti-CD3 (Invitrogen, 16003785) and anti-CD28 (Invitrogen, 16028985) monoclonal antibodies. Upon activation, they were grown in medium containing 10 ng/ml IL-2 (Gibco, PHC0026). All cultures were maintained in 5% CO₂ at 37° C. in a humidified incubator.

### CRISPR/Cas9 Reagents

SpCas9 was purchased from Aldevron (9212-0.25 MG), crRNAs and tracrRNAs were purchased from Integrated DNA Technologies as lyophilized Alt-R® CRISPR/Cas9 products which were then resuspended in nuclease-free water (Invitrogen. AM9937). After resuspension, their concentrations were determined using NanoDrop spectrophotometer (ND-1000). Prior to use, crRNA and tracrRNA were annealed to produce ctRNA. Briefly, crRNA and tracrRNA were mixed at equimolar concentrations, heated at 95° C. for 10 min, then slowly cooled down to RT for 20-30 min for annealing. The final ctRNAs were kept on ice just before use in iTOP reactions. The B2M ctRNA targets the sequence 5′-GAAGTTGACTTACTGAAGAA-3′ (SEQ ID NO: 1), while the CD55 ctRNA targets the sequence 5′-AGGCCGTACAAGTTTTCCCG-3′ (SEQ ID NO: 2).

iTOP in Suspension Cells

0.3 million Jurkat cells or 0.5 million CD4+ T cells were plated on V-bottom 96-well plates (Sigma. M8185) and were centrifuged at 400×g for 3 min at RT. After the removal of supernatants. 50 μl of iTOP mix was added to cells. The mix contained B2M ctRNA, and SpCas9 in 1:1 molar ratio, and NT2100.2. NT2100 or SB (control) buffer (see relevant Examples for details). Following a gentle resuspension of cells in iTOP mix. Jurkat cells were incubated for 45 min, while CD4+ T cells were incubated for 15 min at 37° C. in a humidified incubator with 5% CO₂. At the end of the incubation time. 200 μl culture medium was added to cells, which were then centrifuged at 400×g for 3 min at RT. Following the removal of 200 μl of supernatants, cells were washed once more with 200 μl culture medium to remove the iTOP mix. Finally. cells were resuspended in 200 μl culture medium, transferred to F-bottom 96-plates (Sigma. M3061) and incubated at 37° C. in a humidified incubator with 5% CO₂ until subsequent assays.

iTOP in Adherent Cells

An appropriate number of ARPE-19 cells was plated on F-bottom 96-well plates (Sigma. M3061) to achieve 50% confluency after 24 hours when subjected to iTOP reactions. HEK293 cells were prepared in the same way with the difference that they were plated on 96-well plates (Sigma. M3061) pre-coated with Matrigel (Corning. 356231) in 1:100 dilution in DPBS (Sigma. D8537). hiPSCs were plated 48 hours before iTOP reactions on 96-well plates (FALCON. 353072) pre-coated with Geltrex (Gibco. A1413301). On the day of iTOP, the culture medium was removed and 50 μl iTOP mix was added to cells. The mix for ARPE-19 and HEK293 cells contained B2M ctRNA and SpCas9 in 1:1 molar ratio, and NT2100 or NT2100.2 (see relevant Examples for details). The mix for hiPSCs contained B2M sgRNA or CD55 ctRNA, and SpCas9 in 1:1 molar ratio, and NT2100. NT2100.2 or SB buffer. The incubation time of cells with iTOP mix was as indicated the in relevant examples, at 37° C. in a humidified incubator with 5% CO₂. After incubation. the iTOP mixes were removed and replaced with 200 μl culture medium. Cells were then transferred to a 37° C. humidified incubator with 5% CO₂ until subsequent assays.

### Electroporation

Electroporation was performed with the Nucleofector™ II (Amaxa Biosystems) and the Amaxa® Cell line Nucleofector® Kit V (Lonza) according to the manufacturer's instructions. Briefly. 1 million cells were electroporated in the presence of B2M ctRNA and SpCas9 in 1:1 molar ratio using the X-001 program for Jurkat and ARPE-19 cells. Following electroporation. 0.25 million cells were transferred to F bottom 24-well plates (Sigma. M8812) and were incubated at 37° C. in a humidified incubator with 5% CO₂ until subsequent assays.

### Lipofection

Lipofection was done in F-bottom 96-well plates (Sigma. M3061) with the Lipofectamine™ CRISPRMAX™ Cas9 Transfection Reagent (ThermoFisher Scientific. CMAX00003) according to manufacturer's instructions. The plates for HEK293 cells were pre-coated with Matrigel (Corning. 356231) in 1:100 dilution in DPBS (Sigma. D8537), while the ones for ARPE-19 cells were not. Briefly, ARPE-19 and HEK293 cells at 50% confluency were transfected using lipofectamine in the presence of B2M ctRNA and SpCas9 in 1:1 molar ratio. After lipofection, cells were incubated at 37° C. in a humidified incubator with 5% CO₂ until subsequent assays.

### FACS-Based Recovery and Editing Assays

Depending on the cell type. 4-6 days after iTOP, electroporation, or lipofection cells were used in a FACS based assay to determine their recovery and their loss of MHC-1 or CD55 expression reflecting the levels of B2M and CD55 editing. Suspension Jurkat and CD4+ T cells were transferred to V-bottom 96-well plates (Sigma. M8185), while adherent cells were first detached from F-bottom plates, resuspended in their own pre-collected culture medium and then transferred to V-bottom 96-well plates (Sigma. M8185). ARPE-19 and HEK293 cells were detached using trypsin (Gibco. 25200072), while hiPSCs were detached using 0.5 mM EDTA in DPBS (Sigma, D8537). Following centrifugation at 500×g for 3 min at RT, supernatants were removed, and cells were incubated at 4° C. for 10 min in FACS buffer (5% FBS (Sigma. F7524) in DPBS (Sigma. D8537)) containing 1% of FITC-conjugated anti-human HLA-A,B,C clone W6/32 antibody (Biolegend. 311404) for MHC-1 detection or 2.5% of PE-conjugated anti-human CD55 antibody (Biolegend. 311308) for CD55 detection. To maximize the signal for CD55 detection, cells were also incubated at 4° C. for another 10 min in FACS buffer containing 2.5% of PE-conjugated goat anti-mouse secondary antibody (BD Biosciences. 550589). After incubation with the antibodies, cells were washed twice with plain FACS buffer and resuspended in FACS buffer containing either 0.25% SYTOX Red (Invitrogen. S34859) or 1:5000 DAPI (Invitrogen. D1306) to stain dead cells. Unstained cells and cells killed with 70% ethanol and then stained for dead cells were used for the recovery assay as a negative and positive control, respectively. Control/untreated cells with or without staining with FITC- or PE-conjugated antibodies were used as a negative control for the editing assay. After staining, all cells were transferred to non-coated F-bottom plates (Greiner. 655161) and analyzed using the CytoFlex S flow cytometer (Beckman Coulter). The [640]-660-20A/FSCA channel was used to determine the percentage of SYTOX Red-negative living and SYTOX Red-positive dead cells. The [405]-450-45A/FSC-A channel was used to determine the percentage of DAPI-negative living and DAPI positive dead cells. The recovery was expressed as the percentage of living cells over the total number of cells (sum of living and dead cells). while the debris was excluded from calculations. The [488]-525-40A/FSC-A channel was used to determine the percentage of FITC-negative B2M-edited cells, whereas the [561]-585-42A/FSC-A channel was used to determine the percentage of PE-negative CD55-edited cells.

### FACS-Based Viability Assay

24 hours after iTOP, electroporation, or lipofection the viability of cells was determined with a FACS-based assay. Suspension Jurkat and CD4+ T cells were transferred to V-bottom 96-well plates (Sigma. M8185), while adherent ARPE-19 and HEK293 cells were first detached from F-bottom plates using trypsin (Gibco. 25200072), resuspended in their own pre-collected culture medium and then transferred to V-bottom 96-well plates (Sigma. M8185). Following centrifugation at 500×g for 3 min at RT, supernatants were removed, and cells were resuspended in FACS buffer (5% FBS (Sigma. F7524) in DPBS (Sigma. D8537)) containing 0.25% SYTOX Red dead cell stain (Invitrogen. S34859). Unstained cells and cells killed with 70% ethanol and then stained with SYTOX Red were used as a negative and positive control. respectively. Afterwards, all cells were transferred to non-coated F-bottom plates (Greiner. 655161) and analyzed using the CytoFlex S flow cytometer (Beckman Coulter). The [640]-660-20A/FSC-A channel was used to determine the percentage of living (SYTOX Red-negative) and dead (SYTOX Red-positive) cells. The viability was expressed as the percentage of living cells over the total number of cells (sum of living and dead cells), while the debris was excluded from calculations.

### Cell Lysis and Genotyping

Depending on the cell type. 4-6 days after iTOP, control and iTOPed cells were lysed for subsequent gDNA isolation. B2M or CD55 gene amplification and sequencing, and determination of editing percentage using the Inference of CRISPR Edits (ICE) analysis. Briefly, cells were lysed in lysis buffer (10 mM Tris-HCl pH 7.5, 10 mM EDTA. 10 mM NaCl. 0.5% sarcosyl. 2 mg/ml proteinase K. 0.05 mg/ml RNase) and the generated lysates were incubated overnight at 60° C. gDNA was precipitated from lysates using precipitation solution (75 mM NaCl in pure ethanol), and then it was washed thrice with ethanol, dried, resuspended in nuclease-free water (Invitrogen. AM9937) and used in PCR for B2M or CD55 gene amplification. 5′-TGGCTTGTTGGGAAGGTGGAAG-3′ (SEQ ID NO: 3) forward and 5′-CTGCTGCTCCCTGCTCAAC-(SEQ ID NO: 4) reverse primers were used for B2M amplification, while 5′-CGAAAGGGAGGGCTCAAAGAG-3′ (SEQ ID NO: 5) forward and 5′-CCTGGGGTTTAGTAACGCTAGA-3′ (SEQ ID NO: 6) reverse primers were used for CD55 amplification. Following PCR, the PCR products were analyzed on DNA agarose gel and extracted using the Qiaquick gel extraction kit (Qiagen). The final products were sequenced using the Mix2Seq kit (Eurofins Genomics). The B2M gene was sequenced with 5′-TGGCTTGTTGGGAAGGTGGAAG-3′ (SEQ ID NO: 3) or 5′-GGGAGAAATCGATGACCAAA-3′ (SEQ ID NO: 7) forward primers, whereas the CD55 gene was sequenced with 5′-AAGGGAGGGCTCAAAGAGAC-3′ (SEQ ID NO: 8) forward primer. Finally, the sequences were used in the online tool of ICE analysis (Synthego) (https://icestage.synthego.com/#/) to determine the percentage of B2M and CD55 editing.

1. A method for transducing a protein, nucleic acid or a combination thereof into a cell, wherein the method comprises contacting said cell with the protein or nucleic acid and a transduction buffer, wherein the transduction buffer comprises:
(i) a transduction compound according to formula I: 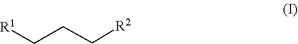
wherein R¹ is selected from SO₃⁻, SO₃H, COOH, COO⁻ or C(O)NR³₂; and
R² is selected from NR³₂, NR³₃⁺, 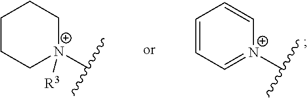
wherein R³ is H or C1-3 alkyl;
(ii) a salt selected from a sodium, rubidium, lithium, potassium or caesium salt, wherein the total concentration of the sodium, rubidium, lithium, potassium or caesium salt is between 500 mM and 1100 mM;
(iii) one or more amino acids selected from serine, asparagine, cysteine, glutamine and threonine, and combinations thereof, wherein the total concentration of amino acids is between 75 mM and 250 mM; and
(iv) one or more disaccharides, wherein the total concentration of disaccharides is between 20 mM and 80 mM.

2. The method of claim 1, wherein the amino acid is serine.

3. The method of any one of the preceding claims, wherein the one or more disaccharides is selected from trehalose, sucrose, lactose, maltose, cellobiose and lactulose, and combinations thereof.

4. The method of any one of the preceding claims, wherein the disaccharide is trehalose.

5. The method of any one of the preceding claims, wherein the salt is sodium chloride.

6. The method of claim 1, wherein the salt is sodium chloride, the amino acid is serine and the disaccharide is trehalose.

7. The method of any one the preceding claims, wherein no rubidium, lithium, potassium or caesium salts are present in the buffer.

8. The method of any one of claims 1-6, wherein sodium chloride is the only sodium, rubidium, lithium, potassium or caesium salt present in a concentration of more than 10 mM.

9. The method of any one of the preceding claims, wherein R¹ is SO₃⁻ or COOH and R² is NR³₂ or

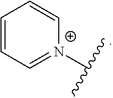 [SMILES: *[n+]1ccccc1]

10. The method of any one of the preceding claims, wherein the transduction compound is selected from compounds 2, 4, 5, 8, 10, 12, 13, 14, 15, 17, 18, 19, 22, 24, 27 and 30 from Table 1.

11. The method of any one of the preceding claims, wherein the transduction compound is GABA or NDSB-201.

12. The method of any one of the preceding claims, wherein the transduction compound is GABA.

13. The method of any one of the preceding claims, wherein the transduction buffer further comprises an osmoprotectant.

14. The method of claim 13, wherein the osmoprotectant is selected from glycerol and/or glycine.

15. The method of claim 13, wherein the osmoprotectant comprises glycerol at a concentration of between 1000 mM and 2000 mM.

16. The method of claim 13, wherein the osmoprotectant comprises glycerol at a concentration of between 1300 mM and 1700 mM.

17. The method of any one of the preceding claims, wherein the concentration of the transduction compound is between 100 mM and 600 mM.

18. The method of any one of the preceding claims, wherein the concentration of the transduction compound is between 200 mM and 300 mM.

19. The method of claim 1, wherein the transduction buffer comprises:
(i) GABA at a concentration of between 200 mM and 300 mM;
(ii) sodium chloride at a concentration of between 750 mM and 850 mM;
(iii) trehalose at a concentration of between 40 mM and 60 mM; and
(iv) serine at a concentration of between 125 mM and 175 mM.

20. The method of claim 1, wherein the transduction buffer comprises:
(i) GABA at a concentration of 250 mM;
(ii) sodium chloride at a concentration of 800 mM;
(iii) trehalose at a concentration of 50 mM; and
(iv) serine at a concentration of 150 mM.

21. The method of claim 19 or claim 20, wherein the transduction buffer further comprises an osmoprotectant, wherein the osmoprotectant comprises glycerol at a concentration of between 1300 mM and 1700 mM.

22. The method of any one of claims 19-21, wherein the cell is an induced pluripotent stem cell or a T cell, preferably a human induced pluripotent stem cell or a primary T cell.

23. The method of claim 22, wherein the protein is a Cas protein, preferably Cas9.

24. The method of any one of the preceding claims, wherein the method comprises contacting the cell with a protein and thereby transducing a protein into the cell.

25. The method of any one of the preceding claims, wherein the protein is a gene editing protein.

26. The method of claim 25, wherein the gene editing protein is selected from zinc finger nucleases, TALENS, Cas proteins, a Cascade complex, a TtAgo protein and an Argonaute protein.

27. The method of claim 25, wherein the gene editing protein is a Cas9, Cas12 or Cas13 protein, preferably a protein selected from selected from SpCas9, SaCas9, FnCas9, NmCas9, TCas9, Cas12a (Cpf1), Cas12b (C2c1), Cas12c (C2c3), Cas13a (C2c2), Cas13b (C2c4), C2c7, Cas13d and dCas9.

28. The method of claim 25, wherein the gene editing protein is a Cas9 protein.

29. The method of claim 28, wherein the gene editing protein is spCas9-1×NLS or 4×NLS-spCas9-2×NLS.

30. The method of claim 28, wherein the gene editing protein is spCas9-1×NLS.

31. The method of claim 25, wherein the gene editing protein is part of a CRISPR-Cas system, optionally selected from CRISPR-Cas9, CRISPR-Prime, CRISPR-dCas9, CRISPR-Cas12, and CRISPR-Cas13.

32. The method of any one of claims 25-31, wherein the protein is added to the transduction buffer at a concentration of between 0.5 μM and 20 μM.

33. The method of any one of claims 25-32, wherein the cell is further contacted with a guide molecule to direct the protein to a target sequence.

34. The method of any one of the preceding claims, wherein the cell is a human cell.

35. The method of any one of the preceding claims, wherein the cell is a stem cell.

36. The method of any one of claims 1-33, wherein the cell is an induced pluripotent stem cell, preferably a human induced pluripotent stem cell.

37. The method of any one of claims 1-34, wherein the cell is a T cell, preferably a primary T cell.

38. The method of claim 36 or claim 37, wherein the protein is a Cas protein, preferably Cas9.

39. The method of any one of the preceding claims, wherein the method is performed in vitro.

40. The method of any one of claims 1-35 or claims 37-38, wherein the method is performed in vivo.

41. A transduction buffer as defined in any one of claims 1-21.

42. The transduction buffer of claim 41, wherein the transduction buffer further comprises the protein, nucleic acid or combination thereof for transduction.

43. A pharmaceutical composition comprising the transduction buffer of claim 41 or claim 42 and a pharmaceutically acceptable carrier.

44. The transduction buffer of claim 41 or claim 42 or the pharmaceutical composition of claim 43, for use in therapy.

45. The transduction buffer of claim 41 or claim 42 the pharmaceutical composition of claim 43, for use in a method of treating a genetic disease, wherein said method comprises contacting a cell with said transduction buffer or pharmaceutical composition and a gene editing protein.

46. The transduction buffer or the pharmaceutical composition for use according to claim 45, wherein the gene editing protein is selected from zinc finger nucleases, TALENS, Cas proteins, a Cascade complex, a TtAgo protein and an Argonaute protein.

47. The transduction buffer or the pharmaceutical composition for use according to claim 45, wherein the gene editing protein is a Cas9 protein.

48. The transduction buffer or the pharmaceutical composition for use according to claim 45, wherein the gene editing protein is spCas9-1×NLS or 4×NLS-spCas9-2×NLS.

49. The transduction buffer or the pharmaceutical composition for use according to claim 45, wherein the gene editing protein is spCas9-1×NLS.

50. The transduction buffer or the pharmaceutical composition for use according to any of claims 45-49, wherein the cell is further contacted with a guide molecule to direct the protein to a target genetic sequence.

51. The transduction buffer or the pharmaceutical composition for use according to any one of claims 45-50, wherein the genetic disease is selected from 22q11.2 deletion syndrome, Angelman syndrome, Canavan disease, Charcot-Marie-Tooth disease, Color blindness, Cri du chat, Cystic fibrosis, Down syndrome, Haemochromatosis, Haemophilia, Klinefelter syndrome, Neurofibromatosis, Phenylketonuria, Polycystic kidney disease, Prader-Willi syndrome, Sickle-cell disease, Tay-Sachs disease, Turner syndrome.

52. The transduction buffer of claim 41 or claim 42 the pharmaceutical composition of claim 43, for use in a method of treating a disease selected from the group consisting of: HIV, Hemophilia B, Mucopolysaccharidosis I, Mucopolysaccharidosis II, Sickle Cell Disease, Thalassemia (such as β-Thalassemia, Thalassemia Major, B-thalassemia Major, and Transfusion Dependent Beta-thalassemia), Leber Congenital Amaurosis 10 (LAC10), Human Papillomavirus-Related Malignant Neoplasm, Acute Myeloid Leukemia, Multiple Myeloma, B-cell Acute Lymphoblastic Leukemia, Metastatic Non-small Cell Lung Cancer, B Cell Leukemia/B Cell Lymphoma, EBV positive advanced stage malignancies, Esophageal Cancer, T cell malignancy, Solid Tumor, Melanoma, Synovial Sarcoma, Liposarcoma, B-cell malignancies, D19⁺ leukemia or lymphoma, Gastro-Intestinal (GI) Cancer, Renal Cell Carcinoma, and Advanced Hepatocellular Carcinoma.


SEQUENCE LISTING (8 sequences)

SEQ ID NO: 1 - DNA, 20 bp, synthetic construct
  misc_feature 1..20: note = B2M ctRNA target sequence
  source 1..20: mol_type = other DNA
gaagttgacttactgaagaa

SEQ ID NO: 2 - DNA, 20 bp, synthetic construct
  misc_feature 1..20: note = CD55 ctRNA target sequence
  source 1..20: mol_type = other DNA
aggccgtacaagttttcccg

SEQ ID NO: 3 - DNA, 22 bp, synthetic construct
  misc_feature 1..22: note = Primer
  source 1..22: mol_type = other DNA
tggcttgttgggaaggtggaag

SEQ ID NO: 4 - DNA, 19 bp, synthetic construct
  misc_feature 1..19: note = Primer
  source 1..19: mol_type = other DNA
ctgctgctccctgctcaac

SEQ ID NO: 5 - DNA, 21 bp, synthetic construct
  misc_feature 1..21: note = Primer
  source 1..21: mol_type = other DNA
cgaaagggagggctcaaagag

SEQ ID NO: 6 - DNA, 22 bp, synthetic construct
  misc_feature 1..22: note = Primer
  source 1..22: mol_type = other DNA
cctggggtttagtaacgctaga

SEQ ID NO: 7 - DNA, 20 bp, synthetic construct
  misc_feature 1..20: note = Primer
  source 1..20: mol_type = other DNA
gggagaaatcgatgaccaaa

SEQ ID NO: 8 - DNA, 20 bp, synthetic construct
  misc_feature 1..20: note = Primer
  source 1..20: mol_type = other DNA
aagggagggctcaaagagac